SEQUENCE LISTING (29 sequences)

SEQ ID NO: 1 - DNA, 902 bp, Bacillus amyloliquefaciens
  source 1..902: mol_type = genomic DNA
taaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggaca

SEQ ID NO: 2 - DNA, 1547 bp, Bacillus velezensis
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggtagccgtatcggaaggtgcggctggatcacctccttt

SEQ ID NO: 3 - DNA, 411 bp, Bacillus velezensis
  source 1..411: mol_type = genomic DNA
gtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatga

SEQ ID NO: 4 - DNA, 559 bp, Bacillus velezensis
  source 1..559: mol_type = genomic DNA
cttcaccttccgttctgtcaaatactttctttcttatacataagatacggcttttgttaacttttgtcaatacttttccagcctggctattccctttagtcaataaacatactagaatataaatagaccgcgggagggggagagactgtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 5 - DNA, 559 bp, Bacillus velezensis; identical to SEQ ID NO: 4
  source 1..559: mol_type = genomic DNA

SEQ ID NO: 6 - DNA, 559 bp, Bacillus velezensis; identical to SEQ ID NO: 4
  source 1..559: mol_type = genomic DNA

SEQ ID NO: 7 - DNA, 559 bp, Bacillus velezensis; identical to SEQ ID NO: 4
  source 1..559: mol_type = genomic DNA

SEQ ID NO: 8 - DNA, 559 bp, Bacillus velezensis; identical to SEQ ID NO: 4
  source 1..559: mol_type = genomic DNA

SEQ ID NO: 9 - DNA, 559 bp, Bacillus velezensis; identical to SEQ ID NO: 4
  source 1..559: mol_type = genomic DNA

SEQ ID NO: 10 - DNA, 559 bp, Bacillus velezensis; identical to SEQ ID NO: 4
  source 1..559: mol_type = genomic DNA

SEQ ID NO: 11 - DNA, 559 bp, Bacillus velezensis; identical to SEQ ID NO: 4
  source 1..559: mol_type = genomic DNA

SEQ ID NO: 12 - DNA, 559 bp, Bacillus velezensis; identical to SEQ ID NO: 4
  source 1..559: mol_type = genomic DNA

SEQ ID NO: 13 - DNA, 559 bp, Bacillus velezensis; identical to SEQ ID NO: 4
  source 1..559: mol_type = genomic DNA

SEQ ID NO: 14 - DNA, 559 bp, Bacillus velezensis; identical to SEQ ID NO: 4
  source 1..559: mol_type = genomic DNA

SEQ ID NO: 15 - DNA, 558 bp, Bacillus velezensis
  source 1..558: mol_type = genomic DNA
cttcaccttccgttctgtcaaatactttctttcttatacataagatacggcttttgttaacttttgtcaatacttttccagcctggctattccctttagtcaataaaatactagaatataaatagaccgcgggagggggagagactgtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 16 - DNA, 558 bp, Bacillus velezensis
  source 1..558: mol_type = genomic DNA
cttcaccttccgttctgtcaaatactttctttcttatacataagatacggcttttgttaacttttgtcaatacttttccagcctggctattccctttagtcaataacatactagaatataaatagaccgcgggagggggagagactgtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 17 - DNA, 558 bp, Bacillus velezensis; identical to SEQ ID NO: 16
  source 1..558: mol_type = genomic DNA

SEQ ID NO: 18 - DNA, 559 bp, Bacillus velezensis
  source 1..559: mol_type = genomic DNA
cttcaccttccgttctgtcaaatactttctttcttatacataagatacggcttttgttaacttttgtcaatacttttccagcctggctattccctttagtcaataaacataccagaatataaatagaccgcgggagggggagagactgtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 19 - DNA, 558 bp, Bacillus velezensis; identical to SEQ ID NO: 15
  source 1..558: mol_type = genomic DNA

SEQ ID NO: 20 - DNA, 559 bp, Bacillus velezensis
  source 1..559: mol_type = genomic DNA
cttcaccttccgttctgtcaaatactttctttcttatacataagatacggcttttgttaacttttgtcaatacttttccagcctggctattccctttagtcaataaacatactagaatataaatagaccgcgggagggggagagactgtggaaagcttcgggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatgcatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 21 - DNA, 558 bp, Bacillus velezensis; identical to SEQ ID NO: 16
  source 1..558: mol_type = genomic DNA

SEQ ID NO: 22 - DNA, 559 bp, Bacillus velezensis
  source 1..559: mol_type = genomic DNA
cttcaccttccgttctgtcaaatactttctttcttatacataagatacggcttttgttaacttttgtcaatacttttccagcctggctattccctttagtcaataaacatactagaatataaatagaccgcgggagggggagagactgtggaaagcttagggaaacaattgcggacattgcgggaaaaaaggcatctgacagtgaaccagctggcaacgtattcaggcgtcagcgccgcgggaatctcgcggattgaaaacggcaaacgcggggtgccgaaacctgcaaccattaaaaagctcgcagatgcgttaaaaatcccgtatgaggaactgatgcagacggcgggatacatagagaccgtccaggaaacgagagccccgtacgacacgggctgcaccttgctggaaaaagccgaaacgtatgagctgcatgatttatccctattggagcgggagaactggcggcacctgtcgaaagaagacttggttctgcttgataaatactttgcgttcttatcggctgaagccaaaaaaagaacaaatgaagactcatgac

SEQ ID NO: 23 - DNA, 558 bp, Bacillus velezensis; identical to SEQ ID NO: 16
  source 1..558: mol_type = genomic DNA

SEQ ID NO: 24 - DNA, 1547 bp, Bacillus methylotrophicus
  source 1..1547: mol_type = genomic DNA
tcggagagtttgatcctggctcaggacgaacgctggcggcgtgcctaatacatgcaagtcgagcggacagatgggagcttgctccctgatgtcagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 25 - DNA, 1547 bp, Bacillus methylotrophicus
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtctgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 26 - DNA, 1547 bp, Bacillus methylotrophicus; identical to SEQ ID NO: 25
  source 1..1547: mol_type = genomic DNA

SEQ ID NO: 27 - DNA, 1547 bp, Bacillus methylotrophicus; identical to SEQ ID NO: 24
  source 1..1547: mol_type = genomic DNA

SEQ ID NO: 28 - DNA, 1547 bp, Bacillus methylotrophicus
  source 1..1547: mol_type = genomic DNA
tcggagagtttgatcctggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

SEQ ID NO: 29 - DNA, 1348 bp, Bacillus methylotrophicus
  source 1..1348: mol_type = genomic DNA
cataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaagtcgtaacaaggtagccgtatcggaaggtgcggctggatcacctccttt

(19) United States
(12) Patent Application Publication
(10) Pub. No.: US 2026/0157377 A1
(43) Pub. Date: Jun. 11, 2026

(54) METHODS AND COMPOSITIONS FOR THE IMPROVEMENT OF MICROBIAL BIOACTIVITY

(71) Applicant: BioConsortia, Inc., Davis, CA (US)

(72) Inventors: Christopher FERRY, Davis, CA (US); Betsy ALFORD, Davis, CA (US); Abhishek PATRI, West Sacramento, CA (US); Debora WILK, Woodland, CA (US); Damian CURTIS, Davis, CA (US)

(21) Appl. No.: 18/706,970

(22) PCT Filed: Nov. 2, 2022
(86) PCT No.: PCT/US2022/079163

§ 371 Date: May 2, 2024

Related U.S. Application Data
(60) Provisional application No. 63/275,372, filed on Nov. 3, 2021.

(51) Int. Cl. A01N 63/22; A01N 63/50; A01P 3/00; A01P 5/00; C12N 1/20; C12N 1/38

(52) U.S. Cl. CPC A01N 63/22; A01N 63/50; A01P 3/00; A01P 5/00; C12N 1/20; C12N 1/38

(57) ABSTRACT

Methods and compositions are provided for improved microbial strains of the taxonomic family Bacillaceae, specifically using such entities as Iturins, fengycins, and/or surfactins. Fermentation conditions are provided that promote formation of different colony morphologies, each of which have different genotypes, gene expression, cyclic lipopeptide production profiles, and bioactivity against particular pests an pathogens.

### CROSS-REFERENCE TO RELATED APPLICATIONS

This application claims the benefit of U.S. Provisional Patent Application Ser. No. 63/275,372 filed 3 Nov. 2021, herein incorporated by reference in its entirety.

### REFERENCE TO SEQUENCE LISTING SUBMITTED ELECTRONICALLY

The official copy of the sequence listing is submitted electronically as an XML sequence listing with a file named 21021-WO-PCT.xml created on 24 Oct. 2022 and having a size of 49,051 bytes and is filed concurrently with the specification. The sequence listing comprised in this XML file is part of the specification and is herein incorporated by reference in its entirety.

### FIELD

The instant disclosure relates generally to the field of biology, specifically microbes and microbial compositions, that may be used in agriculture, pharmaceuticals, cosmetics, bioremediation, animal health, and pest control.

### BACKGROUND

Microbes and compositions they produce are useful in a number of industries, including pharmaceuticals, cosmetics, bioremediation, animal health, cosmetics, and agriculture. A long history of isolating microbial strains has resulted in identification of individual genera, species, and strains for various applications.

It has been observed that some types of microbes exhibit different morphological phenotypes, depending on conditions such as reproductive status (for example, teleomorph and anamorph morphologies of some fungi), nutritional availability (carbon sources), and crowding (quorum sensing). Although some mechanisms of action have been deduced for some types of alternative morphologies, and in some cases a genotypic mutation has been observed that correlates to some types of alternative morphologies, efforts to date have been unsuccessful in producing consistent alternative morphologies that confer benefits for particular applications.

Thus, there remains a need for producing strains of microbes with improved activities, for a variety of applications including agriculture and pharmaceuticals.

### SUMMARY

Provided are methods and compositions for isolated and substantially biologically pure microorganisms of the taxonomic family Bacillaceae that have been cultivated for improved activity, that have application, inter alia, in agriculture, pharmaceuticals, bioremediation, and other fields of use. The disclosed microorganisms can be utilized in their isolated and biologically pure states, as well as being formulated into usable compositions. Further provided are beneficial microbial consortia, comprising at least two members of the disclosed microorganisms, as well as methods of utilizing said consortia in applications.

Various alternative morphologies of Bacillaceae microbe(s) is (are) described, that are the result of one or more genetic variation(s). In some aspects, a “Smooth” bacterial culture morphology is observed, wherein the borders of a colony are substantially unserrated, for example as depicted in FIG. 2A. In some aspects, a “Rough” bacterial culture morphology is observed, wherein the borders of a colony are substantially serrated, for example as depicted in FIG. 2B. In some applications, a type of morphology confers a particular benefit to the microbe or to an organism with which it is associated.

In some aspects, genomic modification of the microbes (individual, consortia, and/or communities) are contemplated, for the improvement of microbial traits and the improvement of microbe-associated organisms and compositions. In some aspects, the genetic variation that causes the phenotypic variation is controlled by altering fermentation conditions and/or provision of an inducible promoter.

### BRIEF DESCRIPTION OF THE DRAWINGS AND THE SEQUENCE LISTING

The disclosure can be more fully understood from the following detailed description and the accompanying drawings and Sequence Listing, all of which form a part of this application.

FIG. 1A depicts the molecular structure of Surfactin.

FIG. 1B depicts the molecular structure of Iturin.

FIG. 1C depicts the molecular structure of Fengycin.

FIG. 2A is a photograph of a Bacillus “Smooth” colony morphology, aerial view of colonies on a plate. Smooth colonies are characterized by a substantially non-serrated surface and margin, and a faint central spot.

FIG. 2B is a photograph of a Bacillus “Rough” colony morphology, aerial view of colonies on a plate. Rough colonies are characterized by a rough surface, erose serrated margin, and a large, dark central spot.

FIG. 3A shows (black box outline) the genomic location of the SNPs identified in Rough Bacillus colony morphologies but not Smooth.

FIG. 3B is a close-up genomic alignment of Rough colony SNPs.

FIG. 4A shows the relative amounts of Iturins, Fengycins, and Surfactins produced by Smooth and Rough Bacillus colonies' Whole Cell Broths (WCBs), as determined by HPLC.

FIG. 4B shows the relative amounts of Iturins, Fengycins, and Surfactins produced by Smooth and Rough Bacillus colonies' Supernatants, as determined by HPLC.

FIG. 5 is a graph of fermentation measurements over time: lipopeptide production (as measured by HPLC, peak area results) on the primary y-axis, and DO₂ (Dissolved Oxygen) levels on the secondary y-axis.

FIG. 6 shows individual lipopeptide (Iturin, Fengycin, and Surfactin) and total lipopeptide production (as measured by HPLC, peak area results) for different fermentation conditions.

FIG. 7 shows control of Squash Powdery Mildew fungal disease severity for different treatments.

FIG. 8 shows control of Apple Scab Disease fungal disease severity for different treatments.

FIG. 9 shows control of Pear Scab Disease fungal disease severity for different treatments.

The sequence descriptions and sequence listing attached hereto comply with the rules governing nucleotide and amino acid sequence disclosures in patent applications as set forth in 37 C.F.R. §§ 1.821 and 1.825. The sequence descriptions comprise the three letter codes for amino acids as defined in 37 C.F.R. §§ 1.821 and 1.825, which are incorporated herein by reference.

SEQID NO: 1 is the Bacillus amyloliquefaciens strain 7084 16S DNA sequence.

SEQID NO: 2 is the Bacillus velezensis strain 20 16S DNA sequence.

SEQID NO: 3 is the hybrid assembly rghR gene sequence DNA sequence from Bacillus velezensis Strain 20.

SEQID NO: 4 is the hybrid assembly variation region sequence DNA sequence from Bacillus velezensis Strain 20.

SEQID NO: 5 is the Smooth variant 1 variation region DNA sequence from Bacillus velezensis Strain 20.

SEQID NO: 6 is the Smooth variant 2 variation region DNA sequence from Bacillus velezensis Strain 20.

SEQID NO: 7 is the Smooth variant 3 variation region DNA sequence from Bacillus velezensis Strain 20.

SEQID NO: 8 is the Smooth variant 4 variation region DNA sequence from Bacillus velezensis Strain 20.

SEQID NO: 9 is the Smooth variant 5 variation region DNA sequence from Bacillus velezensis Strain 20.

SEQID NO: 10 is the Smooth variant 6 variation region DNA sequence from Bacillus velezensis Strain 20.

SEQID NO: 11 is the Smooth variant 7 variation region DNA sequence from Bacillus velezensis Strain 20.

SEQID NO: 12 is the Smooth variant 8 variation region DNA sequence from Bacillus velezensis Strain 20.

SEQID NO: 13 is the Smooth variant 9 variation region DNA sequence from Bacillus velezensis Strain 20.

SEQID NO: 14 is the Smooth variant 10 variation region DNA sequence from Bacillus velezensis Strain 20.

SEQID NO: 15 is the Rough variant 1 variation region DNA sequence from Bacillus velezensis Strain 20.

SEQID NO: 16 is the Rough variant 2 variation region DNA sequence from Bacillus velezensis Strain 20.

SEQID NO: 17 is the Rough variant 3 variation region DNA sequence from Bacillus velezensis Strain 20.

SEQID NO: 18 is the Rough variant 4 variation region DNA sequence from Bacillus velezensis Strain 20.

SEQID NO: 19 is the Rough variant 6 variation region DNA sequence from Bacillus velezensis Strain 20.

SEQID NO: 20 is the Rough variant 7 variation region DNA sequence from Bacillus velezensis Strain 20.

SEQID NO: 21 is the Rough variant 8 variation region DNA sequence from Bacillus velezensis Strain 20.

SEQID NO: 22 is the Rough variant 9 variation region DNA sequence from Bacillus velezensis Strain 20.

SEQID NO: 23 is the Rough variant 10 variation region DNA sequence from Bacillus velezensis Strain 20.

SEQID NO: 24 is the 16S RNA 1 DNA sequence from Bacillus methylotrophicus strain 11604.

SEQID NO: 25 is the 16S RNA 2 DNA sequence from Bacillus methylotrophicus strain 11604.

SEQID NO: 26 is the 16S RNA 3 DNA sequence from Bacillus methylotrophicus strain 11604.

SEQID NO: 27 is the 16S RNA 4 DNA sequence from Bacillus methylotrophicus strain 11604.

SEQID NO: 28 is 16S RNA 5 DNA sequence from Bacillus methylotrophicus strain 11604.

SEQID NO: 29 is the 16S RNA 6 DNA sequence from Bacillus methylotrophicus strain 11604.

### DETAILED DESCRIPTION

Bacterial strains of the taxonomic family Bacillaceae are provided, including strains of the genera Bacillus and Lysinibacillus, that exhibit different colony morphologies, the production of which can depend upon specific culture conditions. Smooth colonies are characterized by a substantially non-serrated surface and margin, and a faint central spot. Rough colonies are characterized by a rough surface, erose serrated margin, and a large, dark central spot.

The inventors determined that Smooth colony phenotypes are predominant when culture conditions are optimized for slower colony growth and other conditions which reduce nutrient stress. Presence of high nutrient media can promote the production of Rough colonies, due to the rapid colony growth phase. Therefore, conditions that pace the growth phase (exponential or logarithmic) of a bacterial culture create conditions that encourage the retention of Smooth colonies and the reduction of production of Rough colonies.

Further, it was observed that Smooth colonies could give rise to Rough colonies, via a mutation in the rghR gene or upstream non-coding sequence region. RghR regulates rapG, rapH, and yvaM by binding to their respective promoter regions (Hayashi et al., Molecular Microbiology 59 (6) 1714-1729; 2006). rghR minus genotypes repress rapG and rapH, which downregulates sfrA, which abolishes the expression of comA and comG.

The Rough colony mutations thus produced a different profile of cyclic lipopeptides, which were less preferred for some applications as compared to the Smooth colony phenotypes.

Rough colony mutants exhibited lower bioactivity against certain pest targets, for example nematodes.

However, it is also contemplated that in some applications, other colony morphologies (e.g., Rough) may be preferred. Culture conditions are provided for the production of Smooth colony morphologies, Rough colony morphologies, as well as mixed cultures of both Rough and Smooth colony morphologies.

Thus, methods and compositions are provided herein for the production and use of Bacillacea colonies that impart benefits to a target, for example to a heterologous cell or heterologous organism, for example to an animal or to a plant.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein the terms “microorganism” or “microbe” should be taken broadly. These terms are used interchangeably and include, but are not limited to, the two prokaryotic domains, Bacteria and Archaea, as well as eukaryotic Fungi and Protists.

As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms) or more than one cell (e.g., multi-cellular microorganism).

As used herein, the term “bacterium” or “bacteria” refers in general to any prokaryotic organism, and may reference an organism from either Kingdom Eubacteria (Bacteria), Kingdom Archaebacteria (Archae), or both. In some cases, bacterial genera or other taxonomic classifications have been reassigned due to various reasons (such as but not limited to the evolving field of whole genome sequencing), and it is understood that such nomenclature reassignments are within the scope of any claimed taxonomy. For example, certain species of the genus Erwinia have been described in the literature as belonging to genus Pantoea (Zhang, Y., Qiu, S.; Examining phylogenetic relationships of Erwinia and Pantoea species using whole genome sequence data. Antonie van Leeuwenhoek 108, 1037-1046 (2015)).

The term “16S” refers to the DNA sequence of the 16S ribosomal RNA (rRNA) sequence of a bacterium. 16S rRNA gene sequencing is a well-established method for studying phylogeny and taxonomy of bacteria.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, J. W. One Fungus=One Name: DNA and fungal nomenclature twenty years after PCR. IMA Fungus 2, 113-120 (2011).). With the development of genomic sequencing, it became evident that taxonomic classification based on molecular phylogenetics did not align with morphological-based nomenclature (Shenoy, B. D., Jeewon, R. and Hyde, K. D. (2007). Impact of DNA sequence-data on the taxonomy of anamorphic fungi. Fungal Diversity 26:1-54.). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, D. L. Managing and coping with names of pleomorphic fungi in a period of transition. IMA Fungus 3, 15-24 (2012)).

The term “Internal Transcribed Spacer” (“ITS”) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (“LSU”) sequence is used to identify fungi. LSU gene sequencing is a well-established method for studying phylogeny and taxonomy of fungi. Some fungal microbes of the present invention may be described by an ITS sequence and some may be described by an LSU sequence. Both are understood to be equally descriptive and accurate for determining taxonomy.

A “population of microorganisms” refers to multiple cells of a single microorganism, in which the cells share common genetic derivation.

The term “microbial consortia” or “microbial consortium” refers to a subset of a microbial community of individual microbial species, or strains of a species, which can be described as carrying out a common function, or can be described as participating in, or leading to, or correlating with, a recognizable parameter or plant phenotypic trait. The community may comprise one or more species, or strains of a species, of microbes. In some instances, the microbes coexist within the community symbiotically.

The term “microbial community” means a group of microbes comprising two or more species or strains. Unlike microbial consortia, a microbial community does not have to be carrying out a common function, or does not have to be participating in, or leading to, or correlating with, a recognizable parameter or plant phenotypic trait.

The term “accelerated microbial selection” or “AMS” is used interchangeably with the term “directed microbial selection” or “DMS” and refers to the iterative selection methodology that was utilized, in some embodiments of the disclosure, to derive the claimed microbial species or consortia of said species.

As used herein, “isolate,” “isolated,” “isolated microbe,” and like terms, are intended to mean that the one or more microorganisms has been separated from at least one of the materials with which it is associated in a particular environment (for example soil, water, plant tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an agricultural carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g., In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a plant. By way of example, the media may be natural or artificial including, but not limited to: soil, potting mixes, bark, vermiculite, hydroponic solutions alone and applied to solid plant support systems, and tissue culture gels. It should be appreciated that the media may be used alone or in combination with one or more other media. It may also be used with or without the addition of exogenous nutrients and physical support systems for roots and foliage.

In one embodiment, the growth medium is a naturally occurring medium such as soil, sand, mud, clay, humus, regolith, rock, or water. In another embodiment, the growth medium is artificial. Such an artificial growth medium may be constructed to mimic the conditions of a naturally occurring medium; however, this is not necessary. Artificial growth media can be made from one or more of any number and combination of materials including sand, minerals, glass, rock, water, metals, salts, nutrients, water. In one embodiment, the growth medium is sterile. In another embodiment, the growth medium is not sterile.

The medium may be amended or enriched with additional compounds or components, for example, a component which may assist in the interaction and/or selection of specific groups of microorganisms with the plant and each other. For example, antibiotics (such as penicillin) or sterilants (for example, quaternary ammonium salts and oxidizing agents) could be present and/or the physical conditions (such as salinity, plant nutrients (for example organic and inorganic minerals (such as phosphorus, nitrogenous salts, ammonia, potassium and micronutrients such as cobalt and magnesium), pH, and/or temperature) could be amended.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant part” is synonymous to a “portion” of a plant, and refers to any part of the plant, and can include distinct tissues and/or organs, and may be used interchangeably with the term “tissue” throughout.

“Progeny” comprises any subsequent generation of an organism, produced via sexual or asexual reproduction.

As used herein, the term “plant element” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Similarly, a “plant reproductive element” is intended to generically reference any part of a plant that is able to initiate other plants via either sexual or asexual reproduction of that plant, for example but not limited to: seed, seedling, root, shoot, cutting, scion, graft, stolon, bulb, tuber, corm, keiki, or bud. The plant element may be in plant or in a plant organ, tissue culture, or cell culture.

The term “monocotyledonous” or “monocot” refers to the subclass of angiosperm plants also known as “monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, the term “cultivar” refers to a variety, strain, or race, of plant that has been produced by horticultural or agronomic techniques and is not normally found in wild populations.

The term “pest” should be taken broadly to encompass any organism or virus that has any type of detrimental effect on another organism. As a few non-limiting examples, pests could include a pathogenic bacterium or fungus, an insect that damages part of a plant or an animal, an herbivore that eats a particular plant, a viral contaminant of a bacterial culture, and/or a nematode that damages part of a plant. Pluralities of individual pests, as well as combinations of different pests, are further contemplated.

As used herein, “improved” should be taken broadly to encompass improvement of a characteristic of a plant, as compared to a control plant, or as compared to a known average quantity associated with the characteristic in question. For example, “improved” plant biomass associated with application of a beneficial microbe, or consortia, of the disclosure can be demonstrated by comparing the biomass of a plant treated by the microbes taught herein to the biomass of a control plant not treated. Alternatively, one could compare the biomass of a plant treated by the microbes taught herein to the average biomass normally attained by the given plant, as represented in scientific or agricultural publications known to those of skill in the art. In the present disclosure, “improved” does not necessarily demand that the data be statistically significant (e.g., p<0.05); rather, any quantifiable difference demonstrating that one value (e.g., the average treatment value) is different from another (e.g., the average control value) can rise to the level of “improved.”

As used herein, “inhibiting and suppressing” and like terms should not be construed to require complete inhibition or suppression, although this may be desired in some embodiments.

As used herein, the term “genotype” refers to the genetic makeup of an individual cell, cell culture, tissue, organism (e.g., a plant), or group of organisms.

As used herein, the term “trait” refers to a characteristic or phenotype. For example, in the context of some embodiments of the present disclosure, yield of a crop relates to the amount of marketable biomass produced by a plant (e.g., fruit, fiber, grain). Desirable traits may also include other plant characteristics, including but not limited to: water use efficiency, nutrient use efficiency, production, mechanical harvestability, fruit maturity, shelf life, pest/disease resistance, early plant maturity, tolerance to stresses, etc. A trait may be inherited in a dominant or recessive manner, or in a partial or incomplete-dominant manner. A trait may be monogenic (i.e., determined by a single locus) or polygenic (i.e., determined by more than one locus) or may also result from the interaction of one or more genes with the environment.

As used herein, the term “phenotype” refers to the observable characteristics of an individual cell, cell culture, organism (e.g., a plant), or group of organisms which results from the interaction between that individual's genetic makeup (i.e., genotype) and the environment.

As used herein, a “synthetic nucleotide sequence” or “synthetic polynucleotide sequence” is a nucleotide sequence that is not known to occur in nature or that is not naturally occurring. Generally, such a synthetic nucleotide sequence will comprise at least one nucleotide difference when compared to any other naturally occurring nucleotide sequence.

The compositions and methods herein may provide for an improved “trait” or “trait of importance” or “trait of interest” to an organism, for example to a plant. For plants, a/an “agronomic trait”, “trait of agronomic importance”, “trait of agronomic interest” may include, but not be limited to, the following: disease resistance, drought tolerance, heat tolerance, cold tolerance, salinity tolerance, metal tolerance, herbicide tolerance, improved water use efficiency, improved nitrogen utilization, improved nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein. “Trait potential” is intended to mean a capability of an organism or element thereof for exhibiting a phenotype, preferably an improved trait, at some point during its life cycle, or conveying said phenotype to another organism or element with which it is associated.

The compositions and methods herein may provide for a “modulated” “agronomic trait” or “trait of agronomic importance” By the term “modulated”, it is intended to refer to a change in an agronomic trait that is changed by virtue of the presence of the microbe(s), exudate, broth, metabolite, etc. In aspects, the modulation provides for the imparting of a beneficial trait.

Such a modulation may be to a property to the host organism (organism with which the microbe is associated), such as a plant, which may include, but not be limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation.

As used herein, the term “molecular marker”, “marker”, or “genetic marker” refers to an indicator that is used in methods for visualizing differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed by the average person skilled in molecular-biological techniques.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue”, “homolog”, or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are Mac Vector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

As used herein, the term “nucleotide change” refers to, e.g., nucleotide substitution, deletion, insertion, chemical alteration, or any of the preceeding, as is well understood in the art.

As used herein, the term “protein modification” refers to, e.g., amino acid substitution, amino acid modification, deletion, and/or insertion, as is well understood in the art.

As used herein, the term “at least a portion” or “fragment” of a nucleic acid or polypeptide means a portion having the minimal size characteristics of such sequences, or any larger fragment of the full-length molecule, up to and including the full length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g., at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na+ ion, typically about 0.01 to 1.0 M Na+ ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g., Ausubel et al., 1998 and Sambrook et al., 2001. In some embodiments, stringent conditions are hybridization in 0.25 M Na₂HPO₄ buffer (pH 7.2) containing 1 mM Na₂EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

In some embodiments, the cell or organism has at least one heterologous trait. As used herein, the term “heterologous trait” refers to a phenotype imparted to a cell or organism by an exogenous molecule or other organism (e.g., a microbe), DNA segment, heterologous polynucleotide or heterologous nucleic acid.

Various changes in phenotype are of interest to the present disclosure, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present disclosure.

A “synthetic combination” can include a combination of a plant and a microbe of the disclosure. The combination may be achieved, for example, by coating the surface of a seed of a plant, such as an agricultural plant, or host plant tissue (root, stem, leaf, etc.), with a microbe of the disclosure. Further, a “synthetic combination” can include a combination of microbes of various strains or species. Synthetic combinations have at least one variable that distinguishes the combination from any combination that occurs in nature. That variable may be, inter alia, a concentration of microbe on a seed or plant tissue that does not occur naturally, or a combination of microbe and plant that does not naturally occur, or a combination of microbes or strains that do not occur naturally together. In each of these instances, the synthetic combination demonstrates the hand of man and possesses structural and/or functional attributes that are not present when the individual elements of the combination are considered in isolation.

In some embodiments, a microbe can be “endogenous” to a seed or plant. As used herein, a microbe is considered “endogenous” to a plant or seed, if the microbe is derived from the plant specimen from which it is sourced. That is, if the microbe is naturally found associated with said plant. In embodiments in which an endogenous microbe is applied to a plant, then the endogenous microbe is applied in an amount that differs from the levels found on the plant in nature. Thus, a microbe that is endogenous to a given plant can still form a synthetic combination with the plant, if the microbe is present on said plant at a level that does not occur naturally.

In some embodiments, a composition (such as a microbe) can be “heterologous” (also termed “exogenous”) to another composition (such as a seed or plant), and in some aspects is referred to herein as a “heterologous composition”. As used herein, a microbe is considered “heterologous” to a plant or seed, if the microbe is not derived from the plant specimen from which it is sourced. That is, if the microbe is not naturally found associated with said plant. For example, a microbe that is normally associated with leaf tissue of a maize plant is considered exogenous to a leaf tissue of another maize plant that naturally lacks said microbe. In another example, a microbe that is normally associated with a maize plant is considered exogenous to a wheat plant that naturally lacks said microbe.

The term “heterologous” may also be applied to relationship of any composition to another, in which the compositions are not naturally found together: non-limiting examples of heterologous relationships include differences in spatial or temporal occurrence, polynucleotide sequences normally found in different organisms or locations within a genome or cell, or a cell that does not normally comprise a particular polynucleotide or polypeptide or does not translate or transcribe such at a particular location or time point.

A composition is “heterologously disposed” when mechanically or manually applied, artificially inoculated, associated with, or disposed onto or into a different cell or organism, for example but not limited plant element, seedling, plant; or onto or into a growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment (for example, drought). In some embodiments, such a manner is contemplated to be selected from the group consisting of: the presence of the microbe; presence of the microbe in a different number of cells, concentration, or amount; the presence of the microbe in a different plant element, tissue, cell type, or other physical location in or on the plant; the presence of the microbe at different time period, e.g., developmental phase of the plant or plant element, time of day, time of season, and combinations thereof. In some embodiments, “heterologously disposed” means that the microbe being applied to a different tissue or cell type of the plant element than that in which the microbe is naturally found. In some embodiments, “heterologously disposed” means that the microbe is applied to a developmental stage of the plant element, seedling, or plant in which said microbe is not naturally associated, but may be associated at other stages. For example, if a microbe is normally found at the flowering stage of a plant and no other stage, a microbe applied at the seedling stage may be considered to be heterologously disposed. In some embodiments, a microbe is heterologously disposed the microbe is normally found in the root tissue of a plant element but not in the leaf tissue, and the microbe is applied to the leaf. In another non-limiting example, if a microbe is naturally found in the mesophyll layer of leaf tissue but is being applied to the epithelial layer, the microbe would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the microbe being applied is at a greater concentration, number, or amount of the plant element, seedling, or plant, than that which is naturally found in said plant element, seedling, or plant. For example, a microbe is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said microbe. In another non-limiting example, a microbe that is naturally found in a tissue of a cupressaceous tree would be considered heterologous to tissue of a maize, wheat, cotton, soybean plant. In another example, a microbe that is naturally found in leaf tissue of a maize, spring wheat, cotton, soybean plant is considered heterologous to a leaf tissue of another maize, spring wheat, cotton, soybean plant that naturally lacks said microbe, or comprises the microbe in a different quantity.

Microbes can also be “heterologously disposed” on a given plant tissue. This means that the microbe is placed upon a plant tissue that it is not naturally found upon. For instance, if a given microbe only naturally occurs on the roots of a given plant, then that microbe could be exogenously applied to the above-ground tissue of a plant and would thereby be “heterologously disposed” upon said plant tissue. As such, a microbe is deemed heterologously disposed, when applied on a plant that does not naturally have the microbe present or does not naturally have the microbe present in the number that is being applied.

### Microbes and Microorganisms

As used herein the term “microorganism” should be taken broadly. It includes, but is not limited to, prokaryotic Bacteria and Archaea, as well as eukaryotic Fungi and Protists.

By way of example, microorganisms may include: Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium, Variovorax and Halomonas), Firmicutes (such as Bacillus, Paenibacillus, Lactobacillus, Mycoplasma, and Acetobacterium), Actinobacteria (such as Brevibacterium, Janibacter, Streptomyces, Rhodococcus, Microbacterium, Curtobacterium, Cellulomonas, and Nocardioides), and the fungi Ascomycota (such as Trichoderma, Ampelomyces, Coniothyrium, Paecoelomyces, Penicillium, Cladosporium, Hypocrea, Beauveria, Metarhizium, Verticullium, Cordyceps, Pichea, and Candida), Basidiomycota (such as Coprinus, Corticium, and Agaricus) and Oomycota (such as Pythium), and Mucoromycota (such as Mucor, and Mortierella); as well as Orbilia/Arthrobotrys, Lysinibacillus, Microbacterium, Talaromyces, Arthrobacter, Kosakonia, Masillia, Novosphingobium, and Tumebacillus.

In a particular embodiment, the microorganism is an endophyte, or an epiphyte, or a microorganism inhabiting the plant rhizosphere or rhizosheath. That is, the microorganism may be found present in the soil material adhered to the roots of a plant or in the area immediately adjacent a plant's roots.

In one embodiment, the microorganism is an endophyte. Endophytes may benefit host plants by preventing pathogenic organisms from colonizing them. Extensive colonization of the plant tissue by endophytes creates a “barrier effect,” where the local endophytes outcompete and prevent pathogenic organisms from taking hold. Endophytes may also produce chemicals which inhibit the growth of competitors, including pathogenic organisms.

In certain embodiments, the microorganism is unculturable. This should be taken to mean that the microorganism is not known to be culturable or is difficult to culture using methods known to one skilled in the art.

Microorganisms of the present disclosure may be collected or obtained from any source or contained within and/or associated with material collected from any source.

In an embodiment, the microorganisms are obtained from any general terrestrial environment, including its soils, plants, fungi, animals (including invertebrates) and other biota, including the sediments, water and biota of lakes and rivers; from the marine environment, its biota and sediments (for example sea water, marine muds, marine plants, marine invertebrates (for example sponges), marine vertebrates (for example, fish)); the terrestrial and marine geosphere (regolith and rock, for example crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, road surfaces).

In another embodiment the microorganisms are collected from a source likely to favor the selection of appropriate microorganisms. By way of example, the source may be a particular environment in which it is desirable for other plants to grow, or which is thought to be associated with terroir. In another example, the source may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the microorganisms may be collected from commercial crops grown in such environments, or more specifically from individual crop plants best displaying a trait of interest amongst a crop grown in any specific environment, for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste, or smell. The microorganisms may be collected from a plant of interest or any material occurring in the environment of interest, including fungi and other animal and plant biota, soil, water, sediments, and other elements of the environment as referred to previously. In certain embodiments, the microorganisms are individual isolates separated from different environments.

In one embodiment, a microorganism or a combination of microorganisms, of use in the methods of the disclosure may be selected from a pre-existing collection of individual microbial species or strains based on some knowledge of their likely or predicted benefit to a plant. For example, the microorganism may be predicted to: improve nitrogen fixation; release phosphate from the soil organic matter; release phosphate from the inorganic forms of phosphate (e.g., rock phosphate); “fix carbon” in the root microsphere; live in the rhizosphere of the plant thereby assisting the plant in absorbing nutrients from the surrounding soil and then providing these more readily to the plant; increase the number of nodules on the plant roots and thereby increase the number of symbiotic nitrogen fixing bacteria (e.g., Rhizobium species) per plant and the amount of nitrogen fixed by the plant; elicit plant defensive responses such as ISR (induced systemic resistance) or SAR (systemic acquired resistance) which help the plant resist the invasion and spread of pathogenic microorganisms; compete with microorganisms deleterious to plant growth or health by antagonism, or competitive utilization of resources such as nutrients or space; change the color of one or more part of the plant, or change the chemical profile of the plant, its smell, taste or one or more other quality.

In one embodiment a microorganism or combination of microorganisms is selected from a pre-existing collection of individual microbial species or strains that provides no knowledge of their likely or predicted benefit to a plant. For example, a collection of unidentified microorganisms isolated from plant tissues without any knowledge of their ability to improve plant growth or health, or a collection of microorganisms collected to explore their potential for producing compounds that could lead to the development of pharmaceutical drugs.

In one embodiment, the microorganisms are acquired from the source material (for example, soil, rock, water, air, dust, plant or other organism) in which they naturally reside. The microorganisms may be provided in any appropriate form, having regard to its intended use in the methods of the disclosure. However, by way of example only, the microorganisms may be provided as an aqueous suspension, gel, homogenate, granule, powder, slurry, live organism or dried material.

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either applied directly to the plant growth medium, or concentrated by filtering or centrifugation, diluted to an appropriate concentration and applied to the plant growth medium with the bulk of the salt removed. By way of further example, microorganisms from mineralized or toxic sources may be similarly treated to recover the microbes for application to the plant growth material to minimize the potential for damage to the plant.

In another embodiment, the microorganisms are used in a crude form, in which they are not isolated from the source material in which they naturally reside. For example, the microorganisms are provided in combination with the source material in which they reside; for example, as soil, or the roots, seed or foliage of a plant. In this embodiment, the source material may include one or more species of microorganisms.

In some embodiments, a mixed population of microorganisms is used in the methods of the disclosure.

In embodiments of the disclosure where the microorganisms are isolated from a source material (for example, the material in which they naturally reside), any one or a combination of a number of standard techniques which will be readily known to skilled persons may be used.

However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

Whilst not essential, in one embodiment, the material containing the microorganisms may be pre-treated prior to the isolation process in order to either multiply all microorganisms in the material, or select portions of the microbial population, either by enriching the material with microbial nutrients or by exposing the sample to low concentrations of an organic solvent or sterilant (for example, household bleach) to enhance the survival of spore-forming or solvent-resistant microorganisms). In one example, pasteurizing the sample to select for microorganisms resistant to heat exposure (for example, bacilli) is contemplated. Microorganisms can then be isolated from the enriched materials or materials treated for selective survival, as above.

In an embodiment of the disclosure, endophytic or epiphytic microorganisms are isolated from plant material. Any number of standard techniques known in the art may be used and the microorganisms may be isolated from any appropriate tissue in the plant, including for example root, stem and leaves, and plant reproductive tissues. By way of example, conventional methods for isolation from plants typically include the sterile excision of the plant material of interest (e.g., root or stem lengths, leaves), surface sterilization with an appropriate solution (e.g., 2% sodium hypochlorite), after which the plant material is placed on nutrient medium for microbial growth (See, for example, Strobel G and Daisy B (2003) Microbiology and Molecular Biology Reviews 67 (4): 491-502; Zinniel D K et al. (2002) Applied and Environmental Microbiology 68 (5): 2198-2208).

In one embodiment of the disclosure, the microorganisms are isolated from root tissue. Further methodology for isolating microorganisms from plant material are detailed hereinafter.

In one embodiment, the microbial population is exposed (prior to the method or at any stage of the method) to a selective pressure. For example, exposure of the microorganisms to pasteurization before their addition to a plant growth medium (preferably sterile) is likely to enhance the probability that the plants selected for a desired trait will be associated with spore-forming microbes that can more easily survive in adverse conditions, in commercial storage, or if applied to seed as a coating, in an adverse environment.

In certain embodiments, as mentioned herein before, the microorganism(s) may be used in crude form and need not be isolated from a plant or a media. For example, plant material or growth media which includes the microorganisms identified to be of benefit to a selected plant may be obtained and used as a crude source of microorganisms for the next round of the method or as a crude source of microorganisms at the conclusion of the method. For example, whole plant material could be obtained and optionally processed, such as mulched or crushed. Alternatively, individual tissues or parts of selected plants (such as leaves, stems, roots, and seeds) may be separated from the plant and optionally processed, such as mulched or crushed. In certain embodiments, one or more part of a plant which is associated with the second set of one or more microorganisms may be removed from one or more selected plants and, where any successive repeat of the method is to be conducted, grafted on to one or more plant used in any step of the plant breeding methods.

### Exemplary Microbes

In some embodiments, the disclosure provides isolated microbial species belonging to genera of: Bacillus, Lysinibacillus.

In some embodiments, a microbe from the genus Bacillus is utilized in agriculture to impart one or more beneficial properties to a plant species.

The isolated microbial species, and novel strains of said species, identified in the present disclosure, are able to impart beneficial properties or traits, such as a trait of agronomic importance, to target plant species.

For instance, the isolated microbes, or consortia of said microbes, are able to improve plant health and vitality. The improved plant health and vitality can be quantitatively measured, for example, by measuring the effect that said microbial application has upon a plant phenotypic or genotypic trait.

### Sourcing, Isolation, Identification, and Culture of Microbes

The microbes of the present disclosure were obtained, among other places, at various locales in New Zealand and the United States

The microbes were identified by utilizing standard microscopic techniques to characterize the microbes' phenotype, which was then utilized to identify the microbe to a taxonomically recognized species.

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for isolated bacteria of the disclosure, biologically pure isolates can be obtained through repeated subculture of biological samples, each subculture followed by streaking onto solid media to obtain individual colonies. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze-dried liquid formulations and cultures stored long term at −70° C. in solutions containing glycerol are contemplated for use in providing formulations of the present inventions.

The bacteria of the disclosure can be propagated in a liquid medium under aerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the bacterial strains include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the bacterial strains, essentially, between 20° C. and 46° C. In some aspects, a temperature range is 30° C.-37° C. For optimal growth, in some embodiments, the medium can be adjusted to pH 7.0-7.4. It will be appreciated that commercially available media may also be used to culture the bacterial strains, such as Nutrient Broth or Nutrient Agar available from Difco, Detroit, MI. It will be appreciated that cultivation time may differ depending on the type of culture medium used and the concentration of sugar as a major carbon source.

In aspects, cultivation lasts between 24-96 hours. Bacterial cells thus obtained are isolated using methods, which are well known in the art. Examples include, but are not limited to, membrane filtration and centrifugal separation. The pH may be adjusted using sodium hydroxide and the like and the culture may be dried using a freeze dryer, until the water content becomes equal to 4% or less. Microbial co-cultures may be obtained by propagating each strain as described hereinabove. It will be appreciated that the microbial strains may be cultured together when compatible culture conditions can be employed.

### Identification of Microbes

Microbes can be distinguished into a genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTm (the difference in the melting temperature between homologous and heterologous hybrids), under standard conditions, are considered to be members of the same species. Thus, populations that share greater than the aforementioned 70% threshold can be considered to be variants of the same species.

For bacterial microbes, the 16S rRNA sequences are often used for determining taxonomy and making distinctions between species, in that if a 16S rRNA sequence shares less than a specified % sequence identity from a reference sequence, then the two organisms from which the sequences were obtained are said to be of different species.

Thus, one could consider microbes to be of the same species, if they share at least 80%, 85%, 90%, 95%, 97%, 98%, or 99% sequence identity across the 16S or 16S rRNA or rDNA sequence. In some aspects, a microbe could be considered to be the same species only if it shares at least 95% identity.

Further, one could define microbial strains of a species, as those that share at least 80%, 85%, 90%, 95%, 97%, 98%, or 99% sequence identity across the 16S rRNA sequence.

Comparisons may also be made with 23S rRNA sequences against reference sequences. In some aspects, a microbe could be considered to be the same strain only if it shares at least 95% identity. In some embodiments, “substantially similar genetic characteristics” means a microbe sharing at least 95% identity.

For fungal microbes, the ITS (Internal Transcriber Sequence) is often used for identification of taxonomy. Among the regions of the ribosomal cistron, the internal transcribed spacer (ITS) region has the highest probability of successful identification for the broadest range of fungi, with the most clearly defined barcode gap between inter- and intraspecific variation, and has been proposed as the formal fungal identification sequence (Schoch et al., PNAS Apr. 17, 2012 109 (16) 6241-6246).

In one embodiment, microbial strains of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-23.

In one embodiment, microbes of the present disclosure include those that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-23.

In one embodiment, microbial consortia of the present disclosure include two or more microbes that comprise polynucleotide sequences that share at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-23.

In one embodiment, microbial consortia of the present disclosure include two or more microbial strains, wherein at least one of those comprises a polynucleotide sequences that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-23.

In one embodiment, microbial consortia of the present disclosure include two or more microbial strains, wherein at least one of those comprises a polynucleotide sequences that shares at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with any one of SEQ ID NOs: 1-23.

Unculturable microbes often cannot be assigned to a definite species in the absence of a phenotype determination, the microbes can be given a candidatus designation within a genus provided their 16S IRNA sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics are made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity and atmospheric conditions, growth in presence of different sole carbon and nitrogen sources. One of ordinary skill in the art would be reasonably apprised as to the phenotypic traits that define the genera of the present disclosure. For instance, colony color, form, and texture on a particular agar (e.g., YMA) was used to identify species of Rhizobium.

In one embodiment, bacterial microbes taught herein were identified utilizing 16S rRNA gene sequences. It is known in the art that 16S rRNA contains hypervariable regions that can provide species/strain-specific signature sequences useful for bacterial identification. In the present disclosure, many of the microbes were identified via partial (500-1200 bp) 16S rRNA sequence signatures. In aspects, each strain represents a pure colony isolate that was selected from an agar plate. Selections were made to represent the diversity of organisms present based on any defining morphological characteristics of colonies on agar medium. The medium used, in embodiments, was R2A, PDA, Nitrogen-free semi-solid medium, or MRS agar. Colony descriptions of each of the ‘picked’ isolates were made after 24-hour growth and then entered into our database. Sequence data was subsequently obtained for each of the isolates.

Phylogenetic analysis using the 16S rRNA gene was used to define “substantially similar” species belonging to common genera and also to define “substantially similar” strains of a given taxonomic species. Further, we recorded physiological and/or biochemical properties of the isolates that can be utilized to highlight both minor and significant differences between strains that could lead to advantageous behavior on plants.

Microbial Populations, Combinations, and/or Consortia

In aspects, the disclosure provides microbial populations, combinations, and/or consortia comprising a combination of at least any two microbes, at least one of which displays a morphology for a preferred application (e.g., Smooth, Rough, or other type).

In certain embodiments, the populations, combinations, and/or consortia of the present disclosure comprise two microbes, or three microbes, or four microbes, or five microbes, or six microbes, or seven microbes, or eight microbes, or nine microbes, or ten or more microbes. Said microbes are different microbial families, genera, species, or strains of microbes.

In some embodiments, the disclosure provides populations, combinations, and/or consortia, comprising: at least one isolated microbial species belonging to family of Bacillaceae.

In some embodiments, the disclosure provides populations, combinations, and/or consortia, comprising: at least one isolated microbial species belonging to genera of: Bacillus or Lysinibacillus.

### Microbial-Produced Compositions

In some cases, the microbes of the present disclosure may produce one or more compounds and/or have one or more activities, e.g., one or more of the following: production of a metabolite, lipopeptide, production of a phytohormone such as auxin, production of acetoin, production of an antimicrobial compound, production of a siderophore, production of a polyketide, production of a phenazine, production of a cellulase, production of a pectinase, production of a chitinase, production of a glucanase, production of a xylanase, nitrogen fixation, or mineral phosphate solubilization.

For example, a microbe of the disclosure may produce a phytohormone selected from the group consisting of an auxin, a cytokinin, a gibberellin, ethylene, a brassinosteroid, and abscisic acid.

Thus, a “metabolite produced by” a microbe of the disclosure, is intended to capture any molecule (small molecule, vitamin, mineral, protein, nucleic acid, lipid, fat, carbohydrate, etc.) produced by the microbe. Often, the exact mechanism of action, whereby a microbe of the disclosure imparts a beneficial trait upon a given plant species is not known. It is hypothesized, that in some instances, the microbe is producing a metabolite that is beneficial to the plant. Thus, in some aspects, a cell-free or inactivated preparation of microbes is beneficial to a plant, as the microbe does not have to be alive to impart a beneficial trait upon the given plant species, so long as the preparation includes a metabolite that was produced by said microbe and which is beneficial to a plant.

In one embodiment, the microbes of the disclosure may produce auxin (e.g., indole-3-acetic acid (IAA)). Production of auxin can be assayed. Many of the microbes described herein may be capable of producing the plant hormone auxin indole-3-acetic acid (IAA) when grown in culture. Auxin plays a key role in altering the physiology of the plant, including the extent of root growth.

Therefore, in an embodiment, the microbes of the disclosure are present as a population disposed on the surface or within a tissue of a given plant species. The microbes may produce a composition, such as a metabolite, in an amount effective to cause a detectable increase in the amount of composition that is found on or within the plant, when compared to a reference plant not treated with the microbes or cell-free or inactive preparations of the disclosure. The composition produced by said microbial population may be beneficial to the plant species.

Such microbial-produced compositions may be present in the cell culture broth or medium/a in which the microbes are grown, or may encompass an exudate produced by the microbes. As used herein, “exudate” refers to one or more compositions excreted by or extracted from one or more microbial cell(s). As used herein, “broth” refers to the collective composition of a cell culture medium after microbial cells are placed in the medium. The composition of the broth may change over time, during different phases of microbial growth and/or development. Broth and/or exudate may improve the traits of plants with which they become associated.

### Lipopeptides

Lipopeptides are microbial surface-active compounds produced by a wide variety of bacteria, fungi, and yeast, characterized by highly diverse structures with a wide array of applications in both therapeutics and agriculture, including as antimicrobials, hemolytics. anti-tumor agents, and antifungals.

Chemically, lipopeptides comprise di-O-acylated-S-(2,3-dihydroxypropyl)-cysteinyl residues N-terminally coupled to different polypeptides. Due to the lipid tail attached to the hydrophilic peptides, lipopeptides have amphiphilic physicochemical properties. The lipid chains of these peptides cause them to self-assemble into nanoparticles such as micelles and fibrils with a core composed of lipidic moieties, while a high density of peptide epitopes is oriented outward.

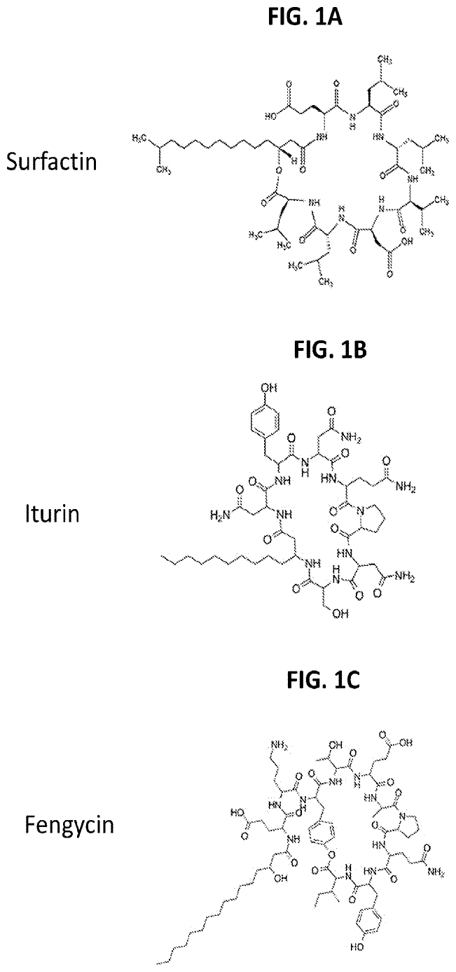

Three major classes of lipopeptides from Bacillus have been described in the literature: surfactins, iturins, and fengycins. Surfactins (example structure in FIG. 1A) comprise seven amino acids linked to one unique hydroxy fatty acid; iturins (example structure in FIG. 1B) comprise seven amino acids linked to one unique amino acid; fengycins (example structure in FIG. 1C) comprise 10 amino acids linked to one unique hydroxy fatty acid. Other classes of lipopeptides are produced in Bacillus (as well as in other organisms) in various quantities.

As discussed in the literature, these lipopeptides are important factors contributing to biocontrol potential in plant growth-promoting microorganisms (see, for example: Malviya et al., Intl J Environmental Research and Public Health Volume 17 Number 1434, 2020).

Different lipopeptides produced by microbes, including cyclolipopeptides, contribute to important phenotypes of the microbe, which can confer benefits to plants with which they are associated.

### Microbial-Induced Traits in Plants

The present disclosure utilizes microbes to impart beneficial properties (or beneficial traits) to desirable plant species, such as agronomic species of interest. In the current disclosure, the terminology “beneficial property” or “beneficial trait” is used interchangeably and denotes that a desirable plant phenotypic or genetic property of interest is modulated, by the application of a microbe or microbial consortia as described herein. As aforementioned, in some aspects, it may very well be that a metabolite produced by a given microbe is ultimately responsible for modulating or imparting a beneficial trait to a given plant.

There are a vast number of beneficial traits that can be modulated by the application of microbes of the disclosure. For instance, the microbes may have the ability to impart one or more beneficial properties to a plant species, for example: increased growth, increased yield, increased nitrogen utilization efficiency, increased stress tolerance, increased drought tolerance, increased photosynthetic rate, enhanced water use efficiency, increased pathogen resistance, modifications to plant architecture that don't necessarily impact plant yield, but rather address plant functionality, causing the plant to increase production of a metabolite of interest, etc.

In aspects, the microbes taught herein provide a wide range of agricultural applications, including: improvements in yield of grain, fruit, and flowers, improvements in growth of plant parts and/or plant elements, improved ability to utilize nutrients (e.g., nitrogen, phosphate, and the like), improved resistance to disease, biopesticidal effects including improved resistance to fungi and nematodes; improved survivability in extreme climate, and improvements in other desired plant phenotypic characteristics.

In some aspects, the isolated microbes, consortia, and/or compositions of the disclosure can be applied to a plant, in order to modulate or alter a plant characteristic such as altered oil content, altered protein content, altered seed carbohydrate composition, altered seed oil composition, altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved nutrient utilization (e.g., phosphate, potassium, and the like), improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, reduced pathogen levels (e.g., via the excretion of metabolites that impair pathogen survival), pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome relative to a reference plant.

In some aspects, the isolated microbes, consortia, and/or compositions of the disclosure can be applied to a plant, in order to modulate in a negative way, a particular plant characteristic. For example, in some aspects, the microbes of the disclosure are able to decrease a phenotypic trait of interest, as this functionality can be desirable in some applications. For instance, the microbes of the disclosure may possess the ability to decrease root growth or decrease root length. Or the microbes may possess the ability to decrease shoot growth or decrease the speed at which a plant grows, as these modulations of a plant trait could be desirable in certain applications.

In some embodiments, the isolated microbes, consortia, and/or compositions of the disclosure can be applied to a plant, in order to impart nematode stress tolerance to plants.

In some embodiments, the isolated microbes, consortia, and/or compositions of the disclosure can be applied to a plant, in order to provide biostimulation (biostimulant effects) to plants.

In some embodiments, the isolated microbes, consortia, and/or compositions of the disclosure can be applied to a plant, in order to provide disease tolerance to plants.

### Compositions

In some embodiments, the microbes of the disclosure are combined with compositions. Compositions generally refer to organic and inorganic compounds that can include compositions that promote the cultivation of the microbe and/or the plant element; compositions involved in formulation of microbes for application to plant elements (for example, but not limited to: wetters, compatibilizing agents (also referred to as “compatibility agents”), antifoam agents, cleaning agents, sequestering agents, drift reduction agents, neutralizing agents and buffers, corrosion inhibitors, dyes, odorants, spreading agents (also referred to as “spreaders”), penetration aids (also referred to as “penetrants”), sticking agents (also referred to as “stickers” or “binders”), dispersing agents, thickening agents (also referred to as “thickeners”), stabilizers, emulsifiers, freezing point depressants, antimicrobial agents, and the like); compositions involved in conferring protection to the plant element or plant (for example, but not limited to: pesticides, nematicides, fungicides, bactericides, herbicides, and the like); as well as other compositions that may be of interest for the particular application.

In some embodiments, the compositions of the present disclosure are solid. Where solid compositions are used, it may be desired to include one or more carrier materials with the active isolated microbe or consortia. In some embodiments, the present disclosure teaches the use of carriers including, but not limited to: mineral earths such as silicas, silica gels, silicates, talc, kaolin, attaclay, limestone, chalk, loess, clay, dolomite, diatomaceous earth, calcium sulfate, magnesium sulfate, magnesium oxide, ground synthetic materials, fertilizers such as ammonium sulfate, ammonium phosphate, ammonium nitrate, thiourea and urea, products of vegetable origin such as cereal meals, tree bark meal, wood meal and nutshell meal, cellulose powders, attapulgites, montmorillonites, mica, vermiculites, synthetic silicas and synthetic calcium silicates, or compositions of these.

### Growth Compositions

In some embodiments, a composition is provided to the microbe and/or the plant element that promotes the growth and development. Exemplary compositions include liquid (such as broth, media) and/or solid (such as soil, nutrients). Various organic or inorganic compounds may be added to the growth composition to facilitate the health of the microbe, alone or in combination with the plant element, for example but not limited to: amino acids, vitamins, minerals, carbohydrates, simple sugars, lipids.

### Formulation Compositions

One or more compositions, in addition to the microbe(s) or microbial-produced composition, may be combined for various application, stability, activity, and/or storage reasons. The additional compositions may be referred to as “formulation component(s)”. Such compositions, or formulation components, may be “heterologous” to a microbe with which they become associated.

In some embodiments, the compositions of the present disclosure are liquid. Thus in some embodiments, the present disclosure teaches that the compositions disclosed herein can include compounds or salts such as monoethanolamine salt, sodium sulfate, potassium sulfate, sodium chloride, potassium chloride, sodium acetate, ammonium hydrogen sulfate, ammonium chloride, ammonium acetate, ammonium formate, ammonium oxalate, ammonium carbonate, ammonium hydrogen carbonate, ammonium thiosulfate, ammonium hydrogen diphosphate, ammonium dihydrogen monophosphate, ammonium sodium hydrogen phosphate, ammonium thiocyanate, ammonium sulfamate or ammonium carbamate.

In some embodiments, the present disclosure teaches that compositions can include binders such as: polyvinylpyrrolidone, polyvinyl alcohol, partially hydrolyzed polyvinyl acetate, carboxymethylcellulose, starch, vinylpyrrolidone/vinyl acetate copolymers and polyvinyl acetate, or compositions of these; lubricants such as magnesium stearate, sodium stearate, talc or polyethylene glycol, or compositions of these; antifoams such as silicone emulsions, long-chain alcohols, phosphoric esters, acetylene diols, fatty acids or organofluorine compounds, and complexing agents such as: salts of ethylenediaminetetraacetic acid (EDTA), salts of trinitrilotriacetic acid or salts of polyphosphoric acids, or compositions of these.

In some embodiments, the compositions comprise surface-active agents. In some embodiments, the surface-active agents are added to liquid compositions. In other embodiments, the surface-active agents are added to solid formulations, especially those to be diluted with a carrier before application. Thus, in some embodiments, the compositions comprise surfactants. Surfactants are sometimes used, either alone or with other additives, such as mineral or vegetable oils as adjuvants to spray-tank mixes to improve the biological performance of the microbes on the target. The types of surfactants used for bioenhancement depend generally on the nature and mode of action of the microbes. The surface-active agents can be anionic, cationic, or nonionic in character, and can be employed as emulsifying agents, wetting agents, suspending agents, or for other purposes. In some embodiments, the surfactants are non-ionics such as: alky ethoxylates, linear aliphatic alcohol ethoxylates, and aliphatic amine ethoxylates. Surfactants conventionally used in the art of formulation and which may also be used in the present formulations are described, in Mccutcheon's Detergents and Emulsifiers Annual, MC Publishing Corp., Ridgewood, N.J., 1998, and in Encyclopedia of Surfactants, Vol. I-III, Chemical Publishing Co., New York, 1980-81. In some embodiments, the present disclosure teaches the use of surfactants including alkali metal, alkaline earth metal or ammonium salts of aromatic sulfonic acids, for example, ligno-, phenol-, naphthalene- and dibutylnaphthalenesulfonic acid, and of fatty acids of arylsulfonates, of alkyl ethers, of lauryl ethers, of fatty alcohol sulfates and of fatty alcohol glycol ether sulfates, condensates of sulfonated naphthalene and its derivatives with formaldehyde, condensates of naphthalene or of the naphthalenesulfonic acids with phenol and formaldehyde, condensates of phenol or phenolsulfonic acid with formaldehyde, condensates of phenol with formaldehyde and sodium sulfite, polyoxyethylene octylphenyl ether, ethoxylated isooctyl-, octyl- or nonylphenol, tributylphenyl polyglycol ether, alkylaryl polyether alcohols, isotridecyl alcohol, ethoxylated castor oil, ethoxylated triarylphenols, salts of phosphated triarylphenolethoxylates, lauryl alcohol polyglycol ether acetate, sorbitol esters, lignin-sulfite waste liquors or methylcellulose, or compositions of these.

In some embodiments, the present disclosure teaches other suitable surface-active agents, including salts of alkyl sulfates, such as diethanolammonium lauryl sulfate; alkylarylsulfonate salts, such as calcium dodecylbenzenesulfonate; alkylphenol-alkylene oxide addition products, such as nonylphenol-C18 ethoxylate; alcohol-alkylene oxide addition products, such as tridecyl alcohol-C16 ethoxylate; soaps, such as sodium stearate; alkylnaphthalene-sulfonate salts, such as sodium dibutyl-naphthalenesulfonate; dialkyl esters of sulfosuccinate salts, such as sodium di(2-ethylhexyl)sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; salts of mono and dialkyl phosphate esters; vegetable oils such as soybean oil, rapeseed/canola oil, olive oil, castor oil, sunflower seed oil, coconut oil, corn oil, cottonseed oil, linseed oil, palm oil, peanut oil, safflower oil, sesame oil, tung oil and the like; and esters of the above vegetable oils, particularly methyl esters.

In some embodiments, the compositions comprise wetting agents. A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank or other vessel to reduce the wetting time of wettable powders and to improve the penetration of water into water-dispersible granules. In some embodiments, examples of wetting agents used in the compositions of the present disclosure, including wettable powders, suspension concentrates, and water-dispersible granule formulations are: sodium lauryl sulphate; sodium dioctyl sulphosuccinate; alkyl phenol ethoxylates; and aliphatic alcohol ethoxylates.

In some embodiments, the compositions of the present disclosure comprise dispersing agents. A dispersing agent is a substance which adsorbs onto the surface of particles and helps to preserve the state of dispersion of the particles and prevents them from re-aggregating. In some embodiments, dispersing agents are added to compositions of the present disclosure to facilitate dispersion and suspension during manufacture, and to ensure the particles redisperse into water in a spray tank. In some embodiments, dispersing agents are used in wettable powders, suspension concentrates, and water-dispersible granules. Surfactants that are used as dispersing agents have the ability to adsorb strongly onto a particle surface and provide a charged or steric barrier to re-aggregation of particles. In some embodiments, the most commonly used surfactants are anionic, non-ionic, or mixtures of the two types.

In some embodiments, for wettable powder formulations, the most common dispersing agents are sodium lignosulphonates. In some embodiments, suspension concentrates provide very good adsorption and stabilization using polyelectrolytes, such as sodium naphthalene sulphonate formaldehyde condensates. In some embodiments, tristyrylphenol ethoxylate phosphate esters are also used. In some embodiments, such as alkylarylethylene oxide condensates and EO-PO block copolymers are sometimes combined with anionics as dispersing agents for suspension concentrates.

In some embodiments, the compositions of the present disclosure comprise polymeric surfactants. In some embodiments, the polymeric surfactants have very long hydrophobic ‘backbones’ and a large number of ethylene oxide chains forming the ‘teeth’ of a ‘comb’ surfactant. In some embodiments, these high molecular weight polymers can give very good long-term stability to suspension concentrates, because the hydrophobic backbones have many anchoring points onto the particle surfaces. In some embodiments, examples of dispersing agents used in compositions of the present disclosure are: sodium lignosulphonates; sodium naphthalene sulphonate formaldehyde condensates; tristyrylphenol ethoxylate phosphate esters; aliphatic alcohol ethoxylates; alky ethoxylates; EO-PO block copolymers; and graft copolymers.

In some embodiments, the compositions of the present disclosure comprise emulsifying agents. An emulsifying agent is a substance, which stabilizes a suspension of droplets of one liquid phase in another liquid phase. Without the emulsifying agent the two liquids would separate into two immiscible liquid phases. In some embodiments, the most commonly used emulsifier blends include alkylphenol or aliphatic alcohol with 12 or more ethylene oxide units and the oil-soluble calcium salt of dodecylbenzene sulphonic acid. A range of hydrophile-lipophile balance (“HLB”) values from 8 to 18 will normally provide good stable emulsions. In some embodiments, emulsion stability can sometimes be improved by the addition of a small amount of an EO-PO block copolymer surfactant.

In some embodiments, the compositions of the present disclosure comprise solubilizing agents. A solubilizing agent is a surfactant, which will form micelles in water at concentrations above the critical micelle concentration. The micelles are then able to dissolve or solubilize water-insoluble materials inside the hydrophobic part of the micelle. The types of surfactants usually used for solubilization are non-ionics: sorbitan monooleates; sorbitan monooleate ethoxylates; and methyl oleate esters.

In some embodiments, the compositions of the present disclosure comprise organic solvents. Organic solvents are used mainly in the formulation of emulsifiable concentrates, ULV formulations, and to a lesser extent granular formulations. Sometimes mixtures of solvents are used. In some embodiments, the present disclosure teaches the use of solvents including aliphatic paraffinic oils such as kerosene or refined paraffins. In other embodiments, the present disclosure teaches the use of aromatic solvents such as xylene and higher molecular weight fractions of C9 and C10 aromatic solvents. In some embodiments, chlorinated hydrocarbons are useful as co-solvents to prevent crystallization of pesticides when the formulation is emulsified into water. Alcohols are sometimes used as co-solvents to increase solvent power.

In some embodiments, the compositions comprise gelling agents. Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. In some embodiments, the compositions comprise one or more thickeners including, but not limited to: montmorillonite, e.g., bentonite; magnesium aluminum silicate; and attapulgite. In some embodiments, the present disclosure teaches the use of polysaccharides as thickening agents. The types of polysaccharides most commonly used are natural extracts of seeds and seaweeds or synthetic derivatives of cellulose. Some embodiments utilize xanthan and some embodiments utilize cellulose. In some embodiments, the present disclosure teaches the use of thickening agents including, but are not limited to: guar gum; locust bean gum; carrageenam; alginates; methyl cellulose; sodium carboxymethyl cellulose (SCMC); hydroxyethyl cellulose (HEC). In some embodiments, the present disclosure teaches the use of other types of anti-settling agents such as modified starches, polyacrylates, polyvinyl alcohol, and polyethylene oxide. Another good anti-settling agent is xanthan gum.

In some embodiments, the presence of surfactants, which lower interfacial tension, can cause water-based formulations to foam during mixing operations in production and in application through a spray tank. Thus, in some embodiments, in order to reduce the tendency to foam, anti-foam agents are often added either during the production stage or before filling into bottles/spray tanks. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

In some embodiments, the compositions comprise a preservative.

In some embodiments, the compositions may be formulated as: a soil drench, a foliar spray, a dip treatment, an in-furrow treatment, a soil amendment, granules, a broadcast treatment, a post-harvest disease control treatment, or a seed treatment. In some embodiments, the compositions may be applied alone in or in rotation spray programs with other agricultural products.

In some embodiments, the compositions may be compatible with tank mixing. In some embodiments, the compositions may be compatible with tank mixing with other agricultural products. In some embodiments, the compositions may be compatible with equipment used for ground, aerial, and irrigation applications.

In some embodiments, the compositions may be applied to genetically modified seeds or plants.

### Protective Compositions

Further, the individual microbes, or microbial consortia, or microbial communities, developed according to the disclosed methods can be combined with known actives available in the agricultural space, such as: pesticide, herbicide, bactericide, fungicide, insecticide, virucide, miticide, nematicide, acaricide, plant growth regulator, rodenticide, anti-algae agent, biocontrol or beneficial agent. Further, the microbes, microbial consortia, or microbial communities developed according to the disclosed methods can be combined with known fertilizers. Such combinations may exhibit synergistic properties. Further still, the individual microbes, or microbial consortia, or microbial communities, developed according to the disclosed methods can be combined with inert ingredients. Also, in some aspects, the disclosed microbes are combined with biological active agents.

In some embodiments, the individual microbes, or microbial consortia, or microbial communities, developed according to the disclosed methods can be combined with biopesticides that function as an herbicide, bactericide, fungicide, insecticide, virucide, miticide, nematicide, acaricide, rodenticide, and/or anti-algae agent. Such biopesticides may be, but are not limited to, macrobial organisms (e.g., beneficial nematodes and the like), microbial organisms (e.g., Serenade, Bt, and the like), plant extracts (e.g., Timorex Gold and the like), biochemical (e.g., insect pheromones and the like), and/or minerals and oils (e.g., canola oil).

### Pesticides and Biopesticides

In some embodiments, the compositions of the present disclosure comprise pesticides, used in combination with the taught microbes. In some embodiments, the compositions of the present disclosure comprise biopesticides, used in combination with the taught microbes.

In some embodiments, the individual microbes, or microbial consortia, or microbial communities, developed according to the disclosed methods can be combined with known pesticides in the agricultural space, such as: pesticides that function as an herbicide, bactericide, fungicide, insecticide, virucide, miticide, nematicide, acaricide, rodenticide, and/or anti-algae agent.

In some embodiments, the individual microbes, or microbial consortia, or microbial communities, developed according to the disclosed methods can be combined with known biopesticides in the agricultural space, such as: biopesticides that function as an herbicide, bactericide, fungicide, insecticide, virucide, miticide, nematicide, acaricide, rodenticide, and/or anti-algae agent.

For example, in some embodiments, the present disclosure teaches compositions comprising one or more of the following active ingredients including: macrobial organisms (e.g., beneficial nematodes and the like), microbial organisms (e.g., Serenade, Bt, and the like), plant extracts (e.g., Timorex Gold and the like), biochemical (e.g., insect pheromones and the like), and/or minerals and oils (e.g., canola oil).

In some embodiments, the individual microbes, or microbial consortia, or microbial communities, developed according to the disclosed methods can be combined with an herbicide selected from the group consisting of: an acetamide selected from the group consisting of acetochlor, alachlor, butachlor, dimethachlor, dimethenamid, flufenacet, mefenacet, metolachlor, metazachlor, napropamide, naproanilide, pethoxamid, pretilachlor, propachlor, and thenylchlor; an amino acid derivative selected from the group consisting of bilanafos, glufosinate, and sulfosate; an aryloxyphenoxypropionate selected from the group consisting of clodinafop, cyhalofop-butyl, fenoxaprop, fluazifop, haloxyfop, metamifop, propaquizafop, quizalofop, and quizalo-fop-P-tefuryl; diquat and paraquat; a (thio) carbamate selected from the group consisting of asulam, butylate, carbetamide, desmedipham, dimepiperate, eptam (EPTC), esprocarb, molinate, orbencarb, phenmedipham, prosulfocarb, pyributicarb, thiobencarb, and triallate; a cyclohexanedione selected from the group consisting of butroxydim, clethodim, cycloxydim, profoxydim, sethoxydim, tepraloxydim, and tralkoxydim; a dinitroaniline selected from the group consisting of benfluralin, ethalfluralin, oryzalin, pendimethalin, prodiamine, and trifluralin; a diphenyl ether selected from the group consisting of acifluorfen, aclonifen, bifenox, diclofop, ethoxyfen, fomesafen, lactofen, and oxyfluorfen; a hydroxybenzonitrile selected from the group consisting of bomoxynil, dichlobenil, and ioxynil; an imidazolinone selected from the group consisting of imazamethabenz, imazamox, imazapic, imazapyr, imazaquin, and imazethapyr; a phenoxy acetic acid selected from the group consisting of clomeprop, 2,4-dichlorophenoxyacetic acid (2,4-D), 2,4-DB, dichlorprop, MCPA, MCPA-thioethyl, MCPB, and Mecoprop; a pyrazine selected from the group consisting of chloridazon, flufenpyr-ethyl, fluthiacet, norflurazon, and pyridate; a pyridine selected from the group consisting of aminopyralid, clopyralid, diflufenican, dithiopyr, fluridone, fluroxypyr, picloram, picolinafen, and thiazopyr; a sulfonyl urea selected from the group consisting of amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, and 14 (2-chloro-6-propyl-imidazol[1,2]-blpyridazin-3-yl)sulfonyl)-3-(4,6-dimethoxy-pyrimidin-2-yl)urea; a triazine selected from the group consisting of ametryn, atrazine, cyanazine, a dimethametryn, ethiozin, hexazinone, metamitron, metribuzin, prometryn, simazine, terbuthylazine, terbutryn, and triaziflam; a urea compound selected from the group consisting of chlorotoluron, daimuron, diuron, fluometuron, isoproturon, linuron, methabenzthiazuron, and tebuthiuron; an acetolactate synthase inhibitor selected from the group consisting of bispyribac-sodium, cloransulam-methyl, diclosulam, florasulam, flucarbazone, flumetsulam, metosulam, ortho-sulfamuron, penoxsulam, propoxycarbazone, pyribambenz-propyl, pyribenzoxim, pyriftalid, pyriminobac-methyl, pyrimisulfan, pyrithiobac, pyroxasulfone, and pyroxsulam; and a compound selected from the group consisting of amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, flurochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclol[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxyl]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

In some embodiments, the individual microbes, or microbial consortia, or microbial communities, developed according to the disclosed methods can be combined with an insecticide selected from the group consisting of: an organo(thio)phosphate selected from the group consisting of acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, and trichlorfon; a carbamate selected from the group consisting of alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, and triazamate; a pyrethroid selected from the group consisting of allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, taufluvalinate, tefluthrin, tetramethrin, tralomethrin, transfluthrin, profluthrin, and dimefluthrin; an insect growth regulator selected from the group consisting of a) a chitin synthesis inhibitor wherein said chitin synthesis inhibitor is a benzoylurea selected from the group consisting of chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, and clofentazine; b) an ecdysone antagonist selected from the group consisting of halofenozide, methoxyfenozide, tebufenozide, and azadirachtin; c) a juvenoid selected from the group consisting of pyriproxyfen, methoprene, and fenoxycarb; or d) a lipid biosynthesis inhibitor selected from the group consisting of spirodiclofen, spiromesifen, and spirotetramat; a nicotinic receptor agonist/antagonist compound selected from the group consisting of clothianidin, dinotefuran, imidacloprid, thiamethoxam, nitenpyram, acetamiprid, thiacloprid, and 1-(2-chloro-thiazol-5-ylmethyl)-2-nitrimino-3,5-dimethyl-[1,3,5]triazinane; a GABA antagonist compound selected from the group consisting of endosulfan, ethiprole, fipronil, vaniliprole, pyrafluprole, pyriprole, and 5-amino-1-(2,6-dichloro-4-methyl-phenyl)-4-sulfinamoyl-1H-pyrazole-3-carbothioic acid amide; a macrocyclic lactone insecticide selected from the group consisting of abamectin, emamectin, milbemectin, lepimectin, spinosad, and spinetoram; a mitochondrial electron transport inhibitor (METI) I acaricide selected from the group consisting of fenazaquin, pyridaben, tebufenpyrad, tolfenpyrad, and flufenerim; a METI II and III compound selected from the group consisting of acequinocyl, fluacyprim, and hydramethylnon; chlorfenapyr; an oxidative phosphorylation inhibitor selected from the group consisting of cyhexatin, diafenthiuron, fenbutatin oxide, and propargite; cryomazine; piperonyl butoxide; a sodium channel blocker selected from the group consisting of indoxacarb and metaflumizone; and a compound selected from the group consisting of benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluron, and pyrifluquinazon.

In some embodiments, the present invention teaches a synergistic use of the presently disclosed microbes or microbial consortia with known pesticides in the agricultural space, such as: pesticides that function as an herbicide, bactericide, fungicide, insecticide, virucide, miticide, nematicide, acaricide, rodenticide, and/or anti-algae agent.

In some embodiments, the present invention teaches a synergistic use of the presently disclosed microbes or microbial consortia with known biopesticides in the agricultural space, such as: biopesticides that function as an herbicide, bactericide, fungicide, insecticide, virucide, miticide, nematicide, acaricide, rodenticide, and/or anti-algae agent.

In some embodiments, when the microbe or microbial consortia identified according to the taught methods is combined with a pesticide one witnesses an additive effect on a plant phenotypic trait of interest. In other embodiments, when the microbe or microbial consortia identified according to the taught methods is combined with a pesticide one witness a synergistic effect on a plant phenotypic trait of interest.

In some embodiments, when the microbe or microbial consortia identified according to the taught methods is combined with a biopesticide one witnesses an additive effect on a plant phenotypic trait of interest. In other embodiments, when the microbe or microbial consortia identified according to the taught methods is combined with a biopesticide one witness a synergistic effect on a plant phenotypic trait of interest.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agriculturally active pesticide compounds and also agricultural auxiliary pesticide compounds.

The isolated microbes and consortia of the present disclosure can synergistically increase the effectiveness of agriculturally active biopesticide compounds and also agricultural auxiliary biopesticide compounds.

### Plant Growth Regulators and Biostimulants

In some embodiments, the compositions of the present disclosure comprise plant growth regulators and/or biostimulants, used in combination with the taught microbes.

In some embodiments, the individual microbes, or microbial consortia, or microbial communities, developed according to the disclosed methods can be combined with known plant growth regulators in the agricultural space, such as: auxins, gibberellins, cytokinins, ethylene generators, growth inhibitors, and growth retardants.

For example, in some embodiments, the present disclosure teaches compositions comprising one or more of the following active ingredients including: ancymidol, butralin, alcohols, chloromequat chloride, cytokinin, daminozide, ethepohon, flurprimidol, giberrelic acid, gibberellin mixtures, indole-3-butryic acid (IBA), maleic hydrazide, mefludide, mepiquat chloride, mepiquat pentaborate, naphthalene-acetic acid (NAA), 1-napthaleneacetemide, (NAD), n-decanol, placlobutrazol, prohexadione calcium, trinexapac-ethyl, uniconazole, salicylic acid, abscisic acid, ethylene, brassinosteroids, jasmonates, polyamines, nitric oxide, strigolactones, or karrikins among others.

In some embodiments, the individual microbes, or microbial consortia, or microbial communities, developed according to the disclosed methods can be combined with seed inoculants known in the agricultural space, such as: QUICKROOTS®, VAULT®, RHIZO-STICK®, NODULATOR®, DORMAL®, SABREX®, among others. In some embodiments, a Bradyrhizobium inoculant is utilized in combination with any single microbe or microbial consortia disclosed here. In particular aspects, a synergistic effect is observed when one combines one of the aforementioned inoculants, e.g., QUICKROOTS® or Bradyrhizobium, with a microbe or microbial consortia as taught herein.

In some embodiments, the compositions of the present disclosure comprise a plant growth regulator, which has: kinetin, gibberellic acid, and indole butyric acid, along with copper, manganese, and zinc.

In some embodiments, the present disclosure teaches compositions comprising one or more commercially available plant growth regulators, including but not limited to: Abide®, A-Rest®, Butralin®, Fair®, Royaltac M®, Sucker-Plucker®, Off-Shoot®, Contact-85®, Citadel®, Cycocel®, E-Pro®, Conklin®, Culbac®, Cytoplex®, Early Harvest®, Foli-Zyme®, Goldengro®, Happygro®, Incite®, Megagro®, Ascend®, Radiate®, Stimulate®, Suppress®, Validate®, X-Cyte®, B-Nine®, Compress®, Dazide®, Boll Buster®, BollD®, Cerone®, Cotton Quik®, Ethrel®, Finish®, Flash®, Florel®, Mature®, MFX®, Prep®, Proxy®, Quali-Pro®, SA-50®, Setup®, Super Boll®, Whiteout®, Cutless®, Legacy®, Mastiff®, Topflor®, Ascend®, Cytoplex®, Ascend®, Early Harvest®, Falgro®, Florgib®, Foli-Zyme®, GA3®, GibGro®, Green Sol®, Incite®, N-Large®, PGR IVR, Pro-Gibb®, Release®, Rouse®, Ryzup®, Stimulate®, BVB®, Chrysal®, Fascination®, Procone®, Fair®, Rite-Hite®, Royal®, Sucker Stuff®, Embark®, Sta-Lo®, Pix®, Pentia®, DipN Grow®, Goldengro®, Hi-Yield®, Rootone®, Antac®, FST-7®, Royaltac®, Bonzi®, Cambistat®, Cutdown®, Downsize®, Florazol®, Paclo®, Paczol®, Piccolo®, Profile®, Shortstop®, Trimmit®, Turf Enhancer®, Apogee®, Armor Tech®, Goldwing®, Governor®, Groom®, Legacy®, Primeraone®, Primo®, Provair®, Solace®, T-Nex®, T-Pac®, Concise®, and Sumagic®.

In some embodiments, the present invention teaches a synergistic use of the presently disclosed microbes or microbial consortia with plant growth regulators and/or stimulants such as phytohormones or chemicals that influence the production or disruption of plant growth regulators.

In some embodiments, the present invention teaches that phytohormones can include: Auxins (e.g., Indole acetic acid IAA), Gibberellins, Cytokinins (e.g., Kinetin), Abscisic acid, Ethylene (and its production as regulated by ACC synthase and disrupted by ACC deaminase).

In some embodiments, the individual microbes, or microbial consortia, or microbial communities, developed according to the disclosed methods can be combined with biostimulants. Such biostimulants may be, but are not limited to, microbial organisms, plant extracts, seaweeds, acids, biochar, and the like.

In some embodiments, the individual microbes, or microbial consortia, or microbial communities, developed according to the disclosed methods can be combined with fertilizers, which may be organic (e.g., manure, blood, fish, and the like), nitrogen-based (e.g., nitrate, ammonium, urea, and the like), phosphate, and potassium. Such fertilizers may also contain micronutrients including, but not limited to, sulfur, iron, zinc, and the like.

In some embodiments, the present invention teaches additional plant-growth promoting chemicals that may act in synergy with the microbes and microbial consortia disclosed herein, such as: humic acids, fulvic acids, amino acids, polyphenols and protein hydrolysates.

Thus, in some embodiments, the disclosure provides for the application of the taught microbes in combination with Ascend® upon any crop. Further, the disclosure provides for the application of the taught microbes in combination with Ascend® upon any crop and utilizing any method or application rate.

In some embodiments, the present disclosure teaches compositions with biostimulants.

As used herein, the term “biostimulant” refers to any substance that acts to stimulate the growth of microorganisms that may be present in soil or other plant growing medium.

The level of microorganisms in the soil or growing medium is directly correlated to plant health. Microorganisms feed on biodegradable carbon sources, and therefore plant health is also correlated with the quantity of organic matter in the soil. While fertilizers provide nutrients to feed and grow plants, in some embodiments, biostimulants provide biodegradable carbon, e.g., molasses, carbohydrates, e.g., sugars, to feed and grow microorganisms. Unless clearly stated otherwise, a biostimulant may comprise a single ingredient, or a combination of several different ingredients, capable of enhancing microbial activity or plant growth and development, due to the effect of one or more of the ingredients, either acting independently or in combination.

In some embodiments, biostimulants are compounds that produce non-nutritional plant growth responses. In some embodiments, many important benefits of biostimulants are based on their ability to influence hormonal activity. Hormones in plants (phytohormones) are chemical messengers regulating normal plant development as well as responses to the environment. Root and shoot growth, as well as other growth responses are regulated by phytohormones. In some embodiments, compounds in biostimulants can alter the hormonal status of a plant and exert large influences over its growth and health. Thus, in some embodiments, the present disclosure teaches sea kelp, humic acids, fulvic acids, and B Vitamins as common components of biostimulants. In some embodiments, the biostimulants of the present disclosure enhance antioxidant activity, which increases the plant's defensive system. In some embodiments, vitamin C, vitamin E, and amino acids such as glycine are antioxidants contained in biostimulants.

In other embodiments, biostimulants may act to stimulate the growth of microorganisms that are present in soil or other plant growing medium. Prior studies have shown that when certain biostimulants comprising specific organic seed extracts (e.g., soybean) were used in combination with a microbial inoculant, the biostimulants were capable of stimulating growth of microbes included in the microbial inoculant. Thus, in some embodiments, the present disclosure teaches one or more biostimulants that, when used with a microbial inoculant, is capable of enhancing the population of both native microbes and inoculant microbes. For a review of some popular uses of biostimulants, please see Calvo et al., 2014, Plant Soil 383:3-41.

### Combinations of Plant Elements, Microbes, and Compositions

In some embodiments, the present disclosure teaches that the individual microbes, or microbial consortia, or microbial communities, or any combination of the preceding, for example comprising any one or a plurality of microorganisms, may be applied to a plant element, optionally in combination with any agricultural composition, for the improvement of a plant phenotype.

Isolated microbes or communities or consortia (generally “microbes” or “microbe”, interchangeably) may be applied to a heterologous plant element, creating a synthetic combination. Microbes are considered heterologous to a plant element if they are not normally associated with the plant element in nature, or if found, are applied in amounts different than that found in nature. In some embodiments, the microbes may be found naturally in one part of a plant but not another, and introduction of the microbes to another part of the plant is considered a heterologous association.

It is further contemplated that the microbe, either isolated or in combination with a plant or plant element, may be further associated with one or more compositions, such as those described above.

Synthetic combinations of microbes and plant elements, microbes and compositions, and microbes and plant elements and compositions are contemplated (generally “synthetic compositions”, compositions that comprise components not typically found associated in nature).

### Plant Element Treatments

In some embodiments, the present disclosure also concerns the discovery that treating plant elements before they are sown or planted with a combination of one or more of the microbes or compositions of the present disclosure can enhance a desired plant trait, e.g., plant growth, plant health, and/or plant resistance to pests.

Thus, in some embodiments, the present disclosure teaches the use of one or more of the microbes or microbial consortia as plant element treatments. The plant element treatment can be a plant element coating applied directly to an untreated and “naked” plant element. However, the plant element treatment can be a plant element overcoat that is applied to a plant element that has already been coated with one or more previous plant element coatings or plant element treatments. The previous plant element treatments may include one or more active compounds, either chemical or biological, and one or more inert ingredients.

The term “plant element treatment” generally refers to application of a material to a plant element prior to or during the time it is planted in soil. Plant element treatment with microbes, and other compositions of the present disclosure, has the advantages of delivering the treatments to the locus at which the plant elements are planted shortly before germination of the plant element and emergence of a plant element.

In other embodiments, the present disclosure also teaches that the use of plant element treatments minimizes the amount of microbe or agricultural composition that is required to successfully treat the plants, and further limits the amount of contact of workers with the microbes and compositions compared to application techniques such as spraying over soil or over emerging plant element.

Moreover, in some embodiments, the present disclosure teaches that the microbes disclosed herein are important for enhancing the early stages of plant life (e.g., within the first thirty days following emergence of the plant element). Thus, in some embodiments, delivery of the microbes and/or compositions of the present disclosure as a plant element treatment places the microbe at the locus of action at a critical time for its activity.

In some embodiments, the microbial compositions of the present disclosure are formulated as a plant element treatment. In some embodiments, it is contemplated that the plant elements can be substantially uniformly coated with one or more layers of the microbes and/or compositions disclosed herein, using conventional methods of mixing, spraying, or a combination thereof through the use of treatment application equipment that is specifically designed and manufactured to accurately, safely, and efficiently apply plant element treatment products to plant elements. Such equipment uses various types of coating technology such as rotary coaters, drum coaters, fluidized bed techniques, spouted beds, rotary mists, or a combination thereof. Liquid plant element treatments such as those of the present disclosure can be applied via either a spinning “atomizer” disk or a spray nozzle, which evenly distributes the plant element treatment onto the plant element as it moves though the spray pattern. In aspects, the plant element is then mixed or tumbled for an additional period of time to achieve additional treatment distribution and drying.

The plant elements can be primed or unprimed before coating with the microbial compositions to increase the uniformity of germination and emergence. In an alternative embodiment, a dry powder formulation can be metered onto the moving plant element and allowed to mix until completely distributed.

In some embodiments, the plant elements have at least part of the surface area coated with a microbiological composition, according to the present disclosure. In some embodiments, a plant element coat comprising the microbial composition is applied directly to a naked plant element. In some embodiments, a plant element overcoat comprising the microbial composition is applied to a plant element that already has a plant element coat applied thereon. In some aspects, the plant element may have a plant element coat comprising, e.g., clothianidin and/or Bacillus firmus-I-1582, upon which the present composition will be applied on top of, as a plant element overcoat. In some aspects, the taught microbial compositions are applied as a plant element overcoat to plant elements that have already been treated with PONCHO™ VOTIVO™ In some aspects, the plant element may have a plant element coat comprising, e.g., Metalaxyl, and/or clothianidin, and/or Bacillus firmus-I-1582, upon which the present composition will be applied on top of, as a plant element overcoat. In some aspects, the taught microbial compositions are applied as a plant element overcoat to plant elements that have already been treated with ACCELERON™.

In some embodiments, the microorganism-treated plant elements have a microbial spore concentration, or microbial cell concentration, from about: 10\hat{ }2 to 10\hat{ }12, 10\hat{ }2 to 10\hat{ }11, 10\hat{ }2 to 10\hat{ }10, 10\hat{ }2 to 10\hat{ }9, 1\hat{ }02 to 10\hat{ }8, 10\hat{ }2 to 10\hat{ }7, 10\hat{ }2 to 10\hat{ }6, 10\hat{ }2 to 10\hat{ }5, 10\hat{ }2 to 10\hat{ }4, or 10\hat{ }2 to 10\hat{ }3 per plant element.

In some embodiments, the microorganism-treated plant elements have a microbial spore concentration, or microbial cell concentration, from about: 10\hat{ }3 to 10\hat{ }12, 10\hat{ }3 to 10\hat{ }11, 10\hat{ }3 to 10\hat{ }10, 10\hat{ }3 to 10\hat{ }9, 10\hat{ }3 to 10\hat{ }8, 10\hat{ }3 to 10\hat{ }7, 10\hat{ }3 to 10\hat{ }6, 10\hat{ }3 to 10\hat{ }5, or 10\hat{ }3 to 10\hat{ }4 per plant element.

In some embodiments, the microorganism-treated plant elements have a microbial spore concentration, or microbial cell concentration, from about: 10\hat{ }4 to 10\hat{ }12, 10\hat{ }4 to 10\hat{ }11, 10\hat{ }4 to 10\hat{ }10, 10\hat{ }4 to 10\hat{ }9, 10\hat{ }4 to 10\hat{ }8, 10\hat{ }4 to 10\hat{ }7, 10\hat{ }4 to 10\hat{ }6, or 10\hat{ }4 to 10\hat{ }5 per plant element.

In some embodiments, the microorganism-treated plant elements have a microbial spore concentration, or microbial cell concentration, from about: 10\hat{ }5 to 10\hat{ }12, 10\hat{ }5 to 10\hat{ }11, 10\hat{ }5 to 10\hat{ }10, 10\hat{ }5 to 10\hat{ }9, 10\hat{ }5 to 10\hat{ }8, 10\hat{ }5 to 10\hat{ }7, or 10\hat{ }5 to 10\hat{ }6 per plant element.

In some embodiments, the microorganism-treated plant elements have a microbial spore concentration, or microbial cell concentration, from about: 105 to 109 per plant element.

In some embodiments, the microorganism-treated plant elements have a microbial spore concentration, or microbial cell concentration, of at least about: 1×10\hat{ }3, or 1×10\hat{ }4, or 1×10\hat{ }5, or 1×10\hat{ }6, or 1×10\hat{ }7, or 1×10\hat{ }8, or 1×10\hat{ }9 per plant element.

In some embodiments, the amount of one or more of the microbes and/or compositions applied to the plant element depend on the final formulation, as well as size or type of the plant or plant element utilized. In some embodiments, one or more of the microbes are present in about 2% w/w/to about 80% w/w of the entire formulation. In some embodiments, the one or more of the microbes employed in the compositions is about 5% w/w to about 65% w/w, or 10% w/w to about 60% w/w by weight of the entire formulation.

In some embodiments, the plant elements may also have more spores or microbial cells per plant element, such as, for example about 10\hat{ }2, 10\hat{ }3, 10\hat{ }4, 10\hat{ }5, 10\hat{ }6, 10\hat{ }7, 10\hat{ }8, 10\hat{ }9, 10\hat{ }10, 10\hat{ }11, 10\hat{ }12, 10\hat{ }13, 10\hat{ }14, 10\hat{ }15, 10\hat{ }16, or 10\hat{ }17 spores or cells per plant element.

In some embodiments, the plant element coats of the present disclosure can be up to 10 μm, 20 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 740 μm, 750 μm, 760 μm, 770 μm, 780 μm, 790 μm, 800 μm, 810 μm, 820 μm, 830 μm, 840 μm, 850 μm, 860 μm, 870 μm, 880 μm, 890 μm, 900 μm, 910 μm, 920 μm, 930 μm, 940 μm, 950 μm, 960 μm, 970 μm, 980 μm, 990 μm, 1000 μm, 1010 μm, 1020 μm, 1030 μm, 1040 μm, 1050 μm, 1060 μm, 1070 μm, 1080 μm, 1090 μm, 1100 μm, 1110 μm, 1120 μm, 1130 μm, 1140 μm, 1150 μm, 1160 μm, 1170 μm, 1180 μm, 1190 μm, 1200 μm, 1210 μm, 1220 μm, 1230 μm, 1240 μm, 1250 μm, 1260 μm, 1270 μm, 1280 μm, 1290 μm, 1300 μm, 1310 μm, 1320 μm, 1330 μm, 1340 μm, 1350 μm, 1360 μm, 1370 μm, 1380 μm, 1390 μm, 1400 μm, 1410 μm, 1420 μm, 1430 μm, 1440 μm, 1450 μm, 1460 μm, 1470 μm, 1480 μm, 1490 μm, 1500 μm, 1510 μm, 1520 μm, 1530 μm, 1540 μm, 1550 μm, 1560 μm, 1570 μm, 1580 μm, 1590 μm, 1600 μm, 1610 μm, 1620 μm, 1630 μm, 1640 μm, 1650 μm, 1660 μm, 1670 μm, 1680 μm, 1690 μm, 1700 μm, 1710 μm, 1720 μm, 1730 μm, 1740 μm, 1750 μm, 1760 μm, 1770 μm, 1780 μm, 1790 μm, 1800 μm, 1810 μm, 1820 μm, 1830 μm, 1840 μm, 1850 μm, 1860 μm, 1870 μm, 1880 μm, 1890 μm, 1900 μm, 1910 μm, 1920 μm, 1930 μm, 1940 μm, 1950 μm, 1960 μm, 1970 μm, 1980 μm, 1990 μm, 2000 μm, 2010 μm, 2020 μm, 2030 μm, 2040 μm, 2050 μm, 2060 μm, 2070 μm, 2080 μm, 2090 μm, 2100 μm, 2110 μm, 2120 μm, 2130 μm, 2140 μm, 2150 μm, 2160 μm, 2170 μm, 2180 μm, 2190 μm, 2200 μm, 2210 μm, 2220 μm, 2230 μm, 2240 μm, 2250 μm, 2260 μm, 2270 μm, 2280 μm, 2290 μm, 2300 μm, 2310 μm, 2320 μm, 2330 μm, 2340 μm, 2350 μm, 2360 μm, 2370 μm, 2380 μm, 2390 μm, 2400 μm, 2410 μm, 2420 μm, 2430 μm, 2440 μm, 2450 μm, 2460 μm, 2470 μm, 2480 μm, 2490 μm, 2500 μm, 2510 μm, 2520 μm, 2530 μm, 2540 μm, 2550 μm, 2560 μm, 2570 μm, 2580 μm, 2590 μm, 2600 μm, 2610 μm, 2620 μm, 2630 μm, 2640 μm, 2650 μm, 2660 μm, 2670 μm, 2680 μm, 2690 μm, 2700 μm, 2710 μm, 2720 μm, 2730 μm, 2740 μm, 2750 μm, 2760 μm, 2770 μm, 2780 μm, 2790 μm, 2800 μm, 2810 μm, 2820 μm, 2830 μm, 2840 μm, 2850 μm, 2860 μm, 2870 μm, 2880 μm, 2890 μm, 2900 μm, 2910 μm, 2920 μm, 2930 μm, 2940 μm, 2950 μm, 2960 μm, 2970 μm, 2980 μm, 2990 μm, or 3000 μm thick.

In some embodiments, the plant element coats of the present disclosure can be 0.5 mm, 1 mm, 1.5 mm, 2 mm, 2.5 mm, 3 mm, 3.5 mm, 4 mm, 4.5 mm, or 5 mm thick.

In some embodiments, the plant element coats of the present disclosure can be at least 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, or 50% of the uncoated plant element weight.

In some embodiments, the microbial spores and/or cells can be coated freely onto the plant elements or they can be formulated in a liquid or solid composition before being coated onto the plant elements. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, it is contemplated that the solid or liquid microbial compositions of the present disclosure further contain functional agents e.g., activated carbon, nutrients (fertilizers), and other agents capable of improving the germination and quality of the products or a combination thereof.

Plant element coating methods and compositions that are known in the art can be particularly useful when they are modified by the addition of one of the embodiments of the present disclosure. Such coating methods and apparatus for their application are disclosed in, for example: U.S. Pat. Nos. 5,916,029; 5,918,413; 5,554,445; 5,389,399; 4,759,945; 4,465,017, and U.S. patent application Ser. No. 13/260,310, each of which is incorporated by reference herein.

Plant element coating compositions are disclosed in, for example: U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432, each of which is incorporated by reference herein.

In some embodiments, a variety of additives can be added to the plant element treatment formulations comprising the inventive compositions. Binders can be added and include those composed of an adhesive polymer that can be natural or synthetic without phytotoxic effect on the plant element to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arabics; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorants may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, molybdenum and zinc.

A polymer or other dust control agent can be applied to retain the treatment on the plant element surface.

In some specific embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrins; alginates; sugars; molasses; polyvinyl pyrrolidones; polysaccharides; proteins; fats; oils; gum arabics; gelatins; syrups; and starches. More examples can be found in, for example, U.S. Pat. No. 7,213,367, incorporated herein by reference.

Various additives, such as adherents, dispersants, surfactants, and nutrient and buffer ingredients, can also be included in the plant element treatment formulation. Other conventional plant element treatment additives include, but are not limited to: coating agents, wetting agents, buffering agents, and polysaccharides. At least one agriculturally acceptable carrier can be added to the plant element treatment formulation such as water, solids, or dry powders. The dry powders can be derived from a variety of materials such as calcium carbonate, gypsum, vermiculite, talc, humus, activated charcoal, and various phosphorous compounds.

In some embodiments, the plant element coating composition can comprise at least one filler, which is an organic or inorganic, natural or synthetic component with which the active components are combined to facilitate its application onto the plant element. In aspects, the filler is an inert solid such as clays, natural or synthetic silicates, silica, resins, waxes, solid fertilizers (for example ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminium or magnesium silicates.

In some embodiments, the plant element treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the plant element, or alternatively may be added as part of the plant element coating composition of the disclosure.

In some embodiments, the formulation that is used to treat the plant element in the present disclosure can be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation can be about 0.5% to about 99% by weight (w/w), or 5-40%, or as otherwise formulated by those skilled in the art.

As mentioned above, other conventional inactive or inert ingredients can be incorporated into the formulation. Such inert ingredients include, but are not limited to: conventional sticking agents; dispersing agents such as methylcellulose, for example, serve as combined dispersant/sticking agents for use in plant element treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the present disclosure can be found in Mccutcheon's, vol. 1, “Emulsifiers and Detergents,” MC Publishing Company, Glen Rock, N.J., U.S.A., 1996, incorporated by reference herein.

The plant element coating formulations of the present disclosure can be applied to plant elements by a variety of methods, including, but not limited to: mixing in a container (e.g., a bottle or bag), mechanical application, tumbling, spraying, and immersion. A variety of active or inert material can be used for contacting plant elements with microbial compositions according to the present disclosure.

In some embodiments, the amount of the microbes or agricultural composition that is used for the treatment of the plant element will vary depending upon the type of plant element and the type of active ingredients, but the treatment will comprise contacting the plant elements with an agriculturally effective amount of the inventive composition.

As discussed above, an effective amount means that amount of the inventive composition that is sufficient to affect beneficial or desired results. An effective amount can be administered in one or more administrations.

In some embodiments, in addition to the coating layer, the plant element may be treated with one or more of the following ingredients: other pesticides including fungicides and herbicides; herbicidal safeners; fertilizers and/or biocontrol agents. These ingredients may be added as a separate layer or alternatively may be added in the coating layer.

In some embodiments, the plant element coating formulations of the present disclosure may be applied to the plant elements using a variety of techniques and machines, such as fluidized bed techniques, the roller mill method, rotostatic plant element treaters, and drum coaters. Other methods, such as spouted beds may also be useful. The plant elements may be pre-sized before coating. After coating, the plant elements are typically dried and then transferred to a sizing machine for sizing. Such procedures are known in the art.

In some embodiments, the microorganism-treated plant elements may also be enveloped with a film overcoating to protect the coating. Such overcoatings are known in the art and may be applied using fluidized bed and drum film coating techniques.

In other embodiments of the present disclosure, compositions according to the present disclosure can be introduced onto a plant element by use of solid matrix priming. For example, a quantity of an inventive composition can be mixed with a solid matrix material and then the plant element can be placed into contact with the solid matrix material for a period to allow the composition to be introduced to the plant element. The plant element can then optionally be separated from the solid matrix material and stored or used, or the mixture of solid matrix material plus plant element can be stored or planted directly. Solid matrix materials which are useful in the present disclosure include polyacrylamide, starch, clay, silica, alumina, soil, sand, polyurea, polyacrylate, or any other material capable of absorbing or adsorbing the inventive composition for a time and releasing that composition into or onto the plant element. It is useful to make sure that the inventive composition and the solid matrix material are compatible with each other. For example, the solid matrix material should be chosen so that it can release the composition at a reasonable rate, for example over a period of minutes, hours, or days.

In some embodiments, the present disclosure teaches that the individual microbes, or microbial consortia, or microbial communities, developed according to the disclosed methods, and can optionally be combined with any plant biostimulant.

In some embodiments, the present disclosure teaches compositions comprising one or more commercially available biostimulants, including but not limited to: Vitazyme®, Diehard™ Biorush®, Diehard™ Biorush® Fe, Diehard™ Soluble Kelp, Diehard™ Humate SP, Phocon®, Foliar Plus™, Plant Plus™, Accomplish LM®, Titan®, Soil Builder™, Nutri Life, Soil Solution™, Seed Coat™, PercPlus™, Plant Power®, CropKarb®, Thrust™, Fast2Grow®, Baccarat®, and Potente® among others.

In some embodiments, when the microbe or microbial consortia identified according to the taught methods is combined with an active chemical agent one witnesses an additive effect on a plant phenotypic trait of interest. In other embodiments, when the microbe or microbial consortia identified according to the taught methods is combined with an active chemical agent one witness a synergistic effect on a plant phenotypic trait of interest.

In some embodiments, when the microbe or microbial consortia identified according to the taught methods is combined with a fertilizer one witnesses an additive effect on a plant phenotypic trait of interest. In other embodiments, when the microbe or microbial consortia identified according to the taught methods is combined with a fertilizer one witness a synergistic effect on a plant phenotypic trait of interest.

In some embodiments, when the microbe or microbial consortia identified according to the taught methods is combined with a plant growth regulator, one witnesses an additive effect on a plant phenotypic trait of interest. In some embodiments, when the microbe or microbial consortia identified according to the taught methods is combined with a plant growth regulator, one witnesses a synergistic effect. In some aspects, the microbes of the present disclosure are combined with Ascend® and a synergistic effect is observed for one or more phenotypic traits of interest.

In some embodiments, when the microbe or microbial consortia identified according to the taught methods is combined with a biostimulant, one witnesses an additive effect on a plant phenotypic trait of interest. In some embodiments, when the microbe or microbial consortia identified according to the taught methods is combined with a biostimulant, one witnesses a synergistic effect.

The synergistic effect obtained by the taught methods can be quantified according to Colby's formula (i.e., (E)=X+Y−(X*Y/100). See Colby, R. S., “Calculating Synergistic and Antagonistic Responses of Herbicide Combinations,” 1967 Weeds, vol. 15, pp. 20-22, incorporated herein by reference in its entirety. Thus, by “synergistic” is intended a component which, by virtue of its presence, increases the desired effect by more than an additive amount.

The isolated microbes and consortia of the present disclosure can synergistically increase the effectiveness of agricultural active compounds and also agricultural auxiliary compounds.

In other embodiments, when the microbe or microbial consortia identified according to the taught methods is combined with a fertilizer one witnesses a synergistic effect.

Furthermore, in certain embodiments, the disclosure utilizes synergistic interactions to define microbial consortia. That is, in certain aspects, the disclosure combines together certain isolated microbial species, which act synergistically, into consortia that impart a beneficial trait upon a plant, or which are correlated with increasing a beneficial plant trait.

The compositions developed according to the disclosure can be formulated with certain auxiliaries, in order to improve the activity of a known active agricultural compound. This has the advantage that the amounts of active ingredient in the formulation may be reduced while maintaining the efficacy of the active compound, thus allowing costs to be kept as low as possible and any official regulations to be followed. In individual cases, it may also possible to widen the spectrum of action of the active compound since plants, where the treatment with a particular active ingredient without addition was insufficiently successful, can indeed be treated successfully by the addition of certain auxiliaries along with the disclosed microbial isolates and consortia. Moreover, the performance of the active may be increased in individual cases by a suitable formulation when the environmental conditions are not favorable.

Such auxiliaries that can be used in an agricultural composition can be an adjuvant. Frequently, adjuvants take the form of surface-active or salt-like compounds. Depending on their mode of action, they can roughly be classified as modifiers, activators, fertilizers, pH buffers, and the like. Modifiers affect the wetting, sticking, and spreading properties of a formulation. Activators break up the waxy cuticle of the plant and improve the penetration of the active ingredient into the cuticle, both short-term (over minutes) and long-term (over hours). Fertilizers such as ammonium sulfate, ammonium nitrate or urea improve the absorption and solubility of the active ingredient and may reduce the antagonistic behavior of active ingredients. pH buffers are conventionally used for bringing the formulation to an optimal pH.

For further embodiments of compositions of the present disclosure, See “Chemistry and Technology of Agrochemical Formulations,” edited by D. A. Knowles, copyright 1998 by Kluwer Academic Publishers, hereby incorporated by reference.

### Plants and Agronomic Benefits

A wide variety of plants, including those cultivated in agriculture, are capable of receiving benefit from the application of microbes, such as those described herein, including single microbes, consortia, and/or compositions produced therefrom, or comprising any of the preceding. Any number of a variety of different plants, including mosses and lichens and algae, may be used in the methods of the disclosure. In embodiments, the plants have economic, social, or environmental value. For example, the plants may include those used as: food crops, fiber crops, oil crops, in the forestry industry, in the pulp and paper industry, as a feedstock for biofuel production, and as ornamental plants.

In other embodiments, the plants may be economically, socially, or environmentally undesirable, such as weeds. The following is a list of non-limiting examples of the types of plants the methods of the disclosure may be applied to:

### Food Crops

Cereals e.g maize, rice, wheat, barley, sorghum, millet, oats, rye, triticale, and buckwheat;
Leafy vegetables e.g., brassicaceous plants such as cabbages, broccoli, bok choy, rocket; salad greens such as spinach, cress, and lettuce;
Fruiting and flowering vegetables e.g., avocado, sweet corn, artichokes; curcubits e.g., squash, cucumbers, melons, courgettes, pumpkins; solanaceous vegetables/fruits e.g., tomatoes, eggplant, and capsicums;
Podded vegetables e.g., groundnuts, peanuts, peas, soybeans, beans, lentils, chickpea, okra;
Bulbed and stem vegetables e.g., asparagus, celery, Allium crops e.g garlic, onions, and leeks;
Roots and tuberous vegetables e.g., carrots, beet, bamboo shoots, cassava, yams, ginger, Jerusalem artichoke, parsnips, radishes, potatoes, sweet potatoes, taro, turnip, and wasabi;
Sugar crops including sugar beet (Beta vulgaris), sugar cane (Saccharum officinarum);
Crops grown for the production of non-alcoholic beverages and stimulants e.g., coffee, black, herbal, and green teas, cocoa, marijuana, and tobacco;
Fruit crops such as true berry fruits (e.g., kiwifruit, grape, currants, gooseberry, guava, feijoa, pomegranate), citrus fruits (e.g., oranges, lemons, limes, grapefruit), epigynous fruits (e.g., bananas, cranberries, blueberries), aggregate fruit (blackberry, raspberry, boysenberry), multiple fruits (e.g., pineapple, fig), stone fruit crops (e.g., apricot, peach, cherry, plum), pip-fruit (e.g., apples, pears) and others such as strawberries, sunflower seeds;
Culinary and medicinal herbs e.g., rosemary, basil, bay laurel, coriander, mint, dill, Hypericum, foxglove, alovera, rosehips, and cannabis;
Crop plants producing spices e.g., black pepper, cumin cinnamon, nutmeg, ginger, cloves, saffron, cardamom, mace, paprika, masalas, star anise;
Crops grown for the production of nuts e.g., almonds and walnuts, Brazil nut, cashew nuts, coconuts, chestnut, macadamia nut, pistachio nuts; peanuts, pecan nuts;
Crops grown for production of beers, wines and other alcoholic beverages e.g grapes, and hops;
Oilseed crops e.g., soybean, peanuts, cotton, olives, sunflower, sesame, lupin species and brassicaeous crops (e.g., canola/oilseed rape); and, edible fungi e.g., white mushrooms, Shiitake and oyster mushrooms;

### Plants Used in Pastoral Agriculture

Legumes: Trifolium species, Medicago species, and Lotus species; White clover (T.repens); Red clover (T. pratense); Caucasian clover (T. ambigum); subterranean clover (T.subterraneum); Alfalfa/Lucerne (Medicago sativum); annual medics; barrel medic; black medic; Sainfoin (Onobrychis viciifolia); Birdsfoot trefoil (Lotus corniculatus); Greater Birdsfoot trefoil (Lotus pedunculatus);
Seed legumes/pulses including Peas (Pisum sativum), Common bean (Phaseolus vulgaris), Broad beans (Vicia faba), Mung bean (Vigna radiata), Cowpea (Vigna unguiculata), Chick pea (Cicer arietum), Lupins (Lupinus species); Cereals including Maize/corn (Zea mays), Sorghum (Sorghum spp.), Millet (Panicum miliaceum, P. sumatrense), Rice (Oryza sativa indica, Oryza sativa japonica), Wheat (Triticum aestivum), Barley (Hordeum vulgare), Rye (Secale cereale), Triticale (Triticum X Secale), Oats (Avena sativa);
Forage and Amenity grasses: Temperate grasses such as Lolium species; Festuca species; Agrostis spp., Perennial ryegrass (Lolium perenne); hybrid ryegrass (Lolium hybridum); annual ryegrass (Lolium multiflorum), tall fescue (Festuca arundinacea); meadow fescue (Festuca pratensis); red fescue (Festuca rubra); Festuca ovina; Festuloliums (Lolium X Festuca crosses); Cocksfoot (Dactylis glomerata); Kentucky bluegrass Poa pratensis; Poa palustris; Poa nemoralis; Poa trivialis; Poa compresa; Bromus species; Phalaris (Phleum species); Arrhenatherum elatius; Agropyron species; Avena strigosa; Setaria italic;
Tropical grasses such as: Phalaris species; Brachiaria species; Eragrostis species; Panicum species; Bahai grass (Paspalum notatum); Brachypodium species; and, grasses used for biofuel production such as Switchgrass (Panicum virgatum) and Miscanthus species;

### Fiber Crops

Cotton, hemp, jute, coconut, sisal, flax (Linum spp.), New Zealand flax (Phormium spp.); plantation and natural forest species harvested for paper and engineered wood fiber products such as coniferous and broadleafed forest species;

### Tree and Shrub Species Used in Plantation Forestry and Bio-Fuel Crops

Pine (Pinus species); Fir (Pseudotsuga species); Spruce (Picea species); Cypress (Cupressus species); Wattle (Acacia species); Alder (Alnus species); Oak species (Quercus species); Redwood (Sequoiadendron species); willow (Salix species); birch (Betula species); Cedar (Cedurus species); Ash (Fraxinus species); Larch (Larix species); Eucalyptus species; Bamboo (Bambuseae species) and Poplars (Populus species).

### Plants Grown for Conversion to Energy, Biofuels or Industrial Products by Extractive, Biological, Physical or Biochemical Treatment

Oil-producing plants such as oil palm, jatropha, soybean, cotton, linseed; Latex-producing plants such as the Para Rubber tree, Hevea brasiliensis and the Panama Rubber Tree Castilla elastica; plants used as direct or indirect feedstocks for the production of biofuels i.e., after chemical, physical (e.g., thermal or catalytic) or biochemical (e.g., enzymatic pre-treatment) or biological (e.g., microbial fermentation) transformation during the production of biofuels, industrial solvents or chemical products e.g., ethanol or butanol, propane dials, or other fuel or industrial material including sugar crops (e.g., beet, sugar cane), starch producing crops (e.g., C3 and C4 cereal crops and tuberous crops), cellulosic crops such as forest trees (e.g., Pines, Eucalypts) and Graminaceous and Poaceous plants such as bamboo, switch grass, miscanthus; crops used in energy, biofuel or industrial chemical production via gasification and/or microbial or catalytic conversion of the gas to biofuels or other industrial raw materials such as solvents or plastics, with or without the production of biochar (e.g., biomass crops such as coniferous, eucalypt, tropical or broadleaf forest trees, graminaceous and poaceous crops such as bamboo, switch grass, miscanthus, sugar cane, or hemp or softwoods such as poplars, willows; and, biomass crops used in the production of biochar;

### Crops Producing Natural Products Useful for the Pharmaceutical, Agricultural Nutraceutical and Cosmeceutical Industries

Crops producing pharmaceutical precursors or compounds or nutraceutical and cosmeceutical compounds and materials for example, star anise (shikimic acid), Japanese knotweed (resveratrol), kiwifruit (soluble fiber, proteolytic enzymes);

Floricultural, Ornamental and Amenity Plants Grown for their Aesthetic or Environmental Properties

Flowers such as roses, tulips, chrysanthemums;
Ornamental shrubs such as Buxus, Hebe, Rosa, Rhododendron, Hedera
Amenity plants such as Platanus, Choisya, Escallonia, Euphorbia, Carex
Mosses such as sphagnum moss

### Plants Grown for Bioremediation

In certain aspects, the microbes of the present disclosure are applied to hybrid plants to increase beneficial traits of said hybrids. In other aspects, the microbes of the present disclosure are applied to genetically modified plants to increase beneficial traits of said GM plants. The microbes taught herein are able to be applied to hybrids and GM plants and thus maximize the elite genetics and trait technologies of these plants.

It should be appreciated that a plant may be provided in the form of a seed, seedling, cutting, propagule, or any other plant material or tissue capable of growing. In one embodiment the seed may be surface-sterilised with a material such as sodium hypochlorite or mercuric chloride to remove surface-contaminating microorganisms. In one embodiment, the propagule is grown in axenic culture before being placed in the plant growth medium, for example as sterile plantlets in tissue culture.

### Methods of Application

The microorganisms may be applied to a plant, seedling, cutting, propagule, or the like and/or the growth medium containing said plant, using any appropriate technique known in the art.

However, by way of example, an isolated microbe, consortia, or composition comprising the same, and/or a composition produced therefrom, may be applied to a plant, seedling, cutting, propagule, or the like, by spraying, coating, dusting, or any other method known in the art.

In another embodiment, the isolated microbe, consortia, or composition comprising the same may be applied directly to a plant seed prior to sowing.

In another embodiment, the isolated microbe, consortia, or composition comprising the same may applied directly to a plant seed, as a seed coating.

In one embodiment of the present disclosure, the isolated microbe, consortia, or composition comprising the same is supplied in the form of granules, or plug, or soil drench that is applied to the plant growth media.

In other embodiments, the isolated microbe, consortia, or composition comprising the same are supplied in the form of a foliar application, such as a foliar spray or liquid composition. The foliar spray or liquid application may be applied to a growing plant or to a growth media, e.g., soil.

In some embodiments, the isolated microbe, consortia, or composition comprising the same are supplied in a form selected from: a soil drench, a foliar spray, a dip treatment, an in furrow treatment, a soil amendment, granules, a broadcast treatment, a post-harvest disease control treatment, or a seed treatment. In some embodiments, the compositions may be applied alone in or in rotation spray programs.

In some embodiments, the isolated microbe, consortia, or composition comprising the same may be compatible with tank mixing. In some embodiments, the compositions may be compatible with tank mixing with other agricultural products. In some embodiments, the compositions may be compatible with equipment used for grould, aerial, and irrigation applications.

In another embodiment, the isolated microbe, consortia, or composition comprising the same may be formulated into granules and applied alongside seeds during planting. Or the granules may be applied after planting. Or the granules may be applied before planting.

In some embodiments, the isolated microbe, consortia, or composition comprising the same are administered to a plant or growth media as a topical application and/or drench application to improve crop growth, yield, and quality. The topical application may be via utilization of a dry mix or powder or dusting composition or may be a liquid based formulation.

In embodiments, the the isolated microbe, consortia, or composition comprising the same can be formulated as: (1) solutions; (2) wettable powders; (3) dusting powders; (4) soluble powders; (5) emulsions or suspension concentrates; (6) seed dressings or coatings, (7) tablets; (8) water-dispersible granules; (9) water soluble granules (slow or fast release); (10) microencapsulated granules or suspensions; (11) as irrigation components, and (12) a component of fertilizers, pesticides, and other compatible amendments, among others. In in certain aspects, the compositions may be diluted in an aqueous medium prior to conventional spray application. The compositions of the present disclosure can be applied to the soil, plant, seed, rhizosphere, rhizosheath, or other area to which it would be beneficial to apply the microbial compositions. Further still, ballistic methods can be utilized as a means for introducing endophytic microbes.

In aspects, the compositions are applied to the foliage of plants. The compositions may be applied to the foliage of plants in the form of an emulsion or suspension concentrate, liquid solution, or foliar spray. The application of the compositions may occur in a laboratory, growth chamber, greenhouse, or in the field.

In another embodiment, microorganisms may be inoculated into a plant by cutting the roots or stems and exposing the plant surface to the microorganisms by spraying, dipping, or otherwise applying a liquid microbial suspension, or gel, or powder.

In another embodiment, the microorganisms may be injected directly into foliar or root tissue, or otherwise inoculated directly into or onto a foliar or root cut, or else into an excised embryo, or radicle, or coleoptile. These inoculated plants may then be further exposed to a growth media containing further microorganisms; however, this is not necessary.

In other embodiments, particularly where the microorganisms are unculturable, the microorganisms may be transferred to a plant by any one or a combination of grafting, insertion of explants, aspiration, electroporation, wounding, root pruning, induction of stomatal opening, or any physical, chemical or biological treatment that provides the opportunity for microbes to enter plant cells or the intercellular space. Persons of skill in the art may readily appreciate a number of alternative techniques that may be used.

In one embodiment, the microorganisms infiltrate parts of the plant such as the roots, stems, leaves and/or reproductive plant parts (become endophytic), and/or grow upon the surface of roots, stems, leaves and/or reproductive plant parts (become epiphytic) and/or grow in the plant rhizosphere. In one embodiment, the microorganisms form a symbiotic relationship with the plant.

While the invention has been particularly shown and described with reference to a preferred embodiment and various alternate embodiments, it will be understood by persons skilled in the relevant art that various changes in form and details can be made therein without departing from the spirit and scope of the invention. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof.

All cited patents and publications referred to in this application are herein incorporated by reference in their entirety, for all purposes, to the same extent as if each were individually and specifically incorporated by reference. Mention of any reference, article, publication, patent, patent publication, and patent application cited herein is not, however, and should not be taken as, an acknowledgment or any form of suggestion that they constitute valid prior art or form part of the common general knowledge in any country in the world.

### EXAMPLES

In certain culture conditions, bacteria assumed different morphologies. Scale-up and optimization of fermentation conditions for production of bacteria of the family Bacillacea resulted in a greater incidence of a rough-appearing phenotype (“Rough”) that produced fewer lipopeptides and had lower activity against certain types of assay targets, than the preferred phenotype (“Smooth”).

Bacillacea Rough colony morphologies appeared to occur when fermentation conditions promoted fast colony growth and rapid nutrient utilization. These conditions encouraged the colonies to approach end-phase faster, and thus adopted a stress-response phenotype of Rough.

Colonies with Rough phenotype comprised a mutation in the rghR gene, which is an HTH-type transcriptional repressor. Studies have shown (Hayashi et al., 2006) that rghR-strains exhibit depression of rapG and rapH expression, which leads to a down-regulation of sfrA, which abolishes expression of comK and comG.

The inventors developed methods for encouraging Bacillacea strains to assume Smooth morphologies during different fermentation and production methods. The methods disclosed herein promote the production of preferred bacterial phenotypes, with surprisingly high efficacy rates. During fermentation, conditions are provided that encourage the colony morphology ratio in mixed cultures to be as high as 50/50, and other fermentation conditions are provided that promote 100% preferred colony morphology.

### Example 1: Characterization of Bacterial Morphologies

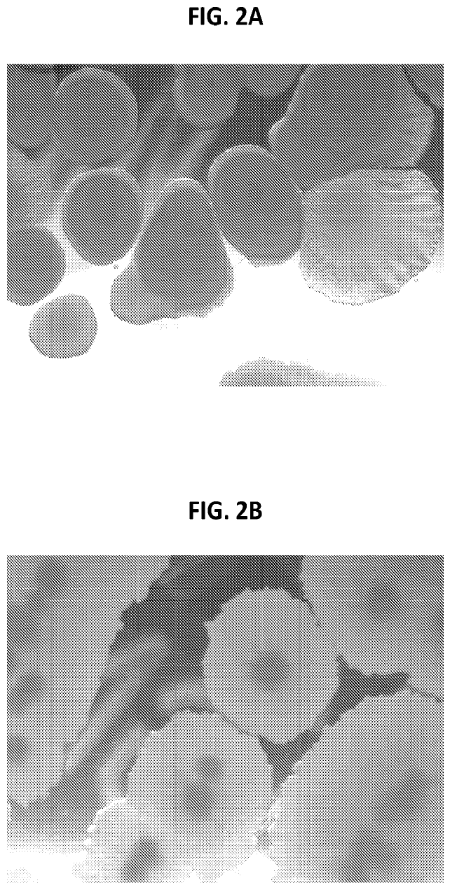

Different Bacillus colony morphologies, as demonstrated by the exemplary strains Bacillus amyloliquefaciens (NRRL Deposit B-67810 deposited on 3 Jul. 2019; 16S given as SEQID NO: 1) (family Bacillaceae) and Bacillus velezensis (16S given as SEQID NO: 2) (family Bacillaceae) appeared as “Smooth” (exemplified in FIG. 2A) or “Rough” (exemplified in FIG. 2B)

TABLE 1
List of selected strains of family Bacillaceae and different morphologies
identified during various fermentation conditions

| | # of different | | |
|---|---|---|---|
| Strain | morphologies | Genomic sequencing taxonomy | 16S taxonomy |
| 7085 | 2 | Bacillus amyloliquefaciens | Bacillus amyloliquefaciens |
| 8110 | 2 | Bacillus velezensis | Bacillus amyloliquefaciens |
| 104624 | 2 | Bacillus megaterium | Bacillus aryabhattai |
| 105146 | 2 | not sequenced | Bacillus circulans |
| 101678 | 2 | Bacillus megaterium | Bacillus megaterium |
| 104908 | 2 | Bacillus megaterium | Bacillus megaterium |
| 105094 | 2 | Bacillus megaterium | Bacillus megaterium |
| 101677 | 2 | | Bacillus megaterium |
| 15765 | 2 | Bacillus pumilus | Bacillus pumilus |
| 9047 | 2 | Bacillus amyloliquefaciens | Bacillus sp. |
| 8342 | 2 | Bacillus megaterium | Bacillus sp. |
| 14135 | 2 | Bacillus subtilis | Bacillus subtilis |
| 104008 | 2 | | Bacillus subtilis |
| 103990 | 2 | | Bacillus subtilis |
| 104012 | 2 | | Bacillus subtilis |
| 7031 | 2 | Bacillus amyloliquefaciens | Bacillus tequilensis |
| 7084 | 2 | Bacillus amyloliquefaciens | Bacillus tequilensis |
| 8012 | 2 | Bacillus subtilis | Bacillus tequilensis |
| 103989 | 2 | | Bacillus tequilensis |
| 103991 | 2 | Bacillus amyloliquefaciens | Bacillus velezensis |
| 103887 | 2 | Bacillus velezensis | Bacillus velezensis |
| 104487 | 2 | Bacillus velezensis | Bacillus velezensis |
| 104594 | 2 | Bacillus velezensis | Bacillus velezensis |
| 20 | 2 | Bacillus velezensis | Bacillus velezensis |
| 103995 | 2 | | Bacillus velezensis |
| 14520 | 2 | Lysinibacillus sp. YS11 | Lysinibacillus fusiformis |
| 11604 | 2-3 | Bacillus methylotrophicus | |

### Example 2: Bacterial Culture and Fermentation Conditions

Different culture conditions produced different morphologies of strains, as demonstrated by the strains listed as examples in Table 1.

Initial cultures on agar plates showed that some strains formed different morphologies. Follow-up experiments were conducted on Strain 20, Strain 7084, and Strain 11604.

Strains 20 and 7084 produced two different colony morphologies (Smooth and Rough) under various fermentation conditions, such as those listed in Table 2.

Strain 11604 produced multiple different colony morphologies when grown on different media: Smooth colonies were seen on R2A agar (a low-nutrient medium), Rough colonies were seen on BEA agar (which inhibits the growth of Gram-positive bacteria), and SB agar plates produced colonies with a reddish color.

Further studies were conducted using Strain 20 as a representative strain. Starting cultures for all conditions below were single morphology (Smooth) at the time of inoculation. Optimized media comprised sucrose, maltose, and soy flour. A summary of fermentation variables is given in Table 2.

### Shake Flask Fermentations

Shake flask batch fermentations in optimized media for at least 4 days at 30 degrees Celsius yielded a Smooth: Rough colony ration of approximately 1:1.

Shake flask batch fermentation in optimized media with a smaller volume (500 mL) in 2 L glass flasks at 3 days yielded a single colony morphology (Smooth). Low CFU (Colony Forming Units) numbers were produced, suggesting poor oxygenation and lower nutrient consumption. Fed-batch studies (below) indicated that nutrient starvation (Carbon) is the likely trigger for increased Rough colony production.

Shake flask fed-batch fermentation in optimized media at 30 degrees Celsius with sucrose feed at 16 hours yielded a single morphology culture (Smooth).

### Bioreactor Fermentations

Bioreactor fermentation in optimized media for at least 48 hours at 30 degrees Celsius yielded a Smooth: Rough ratio of approximately 1:1.

Bioreactor fed-batch fermentations in optimized media at 30 degrees Celsius for at least 48 hours yielded a single morphology culture (Smooth).

### Colony Morphology Lability

Bacillus Smooth colonies were able to give rise to either/both Smooth and/or Rough colonies; however, once a colony became Rough it only gave rise to other Rough colonies.

It is hypothesized that lower oxygenation levels promoted production of the preferred (Smooth) phenotype, as those colonies have lower nutrient utilization under oxygen stress so they do not approach conditions of nutrient stress. Certain stresses, for example nutrient stress, seemed to be a predictor of a Bacillus Smooth colony mutating into a Rough colony phenotype.

As shown in subsequent examples, Smooth and Rough comprised different genotypes, as well as different lipopeptide production profiles. Additionally, Smooth colonies performed better in biocontrol (e.g., nematocidal or fungicidal activity) assays.

TABLE 2
Summary of Bacillus fermentation condition variables and colony morphology(ies)

| | SF-B | SF-B | SF-B | SF-B | SF-FB | BIO-B | BIO-FB |
|---|---|---|---|---|---|---|---|
| Inoculation Source | −80*C. stock | −80*C. stock | −80*C. stock | −80*C. stock | −80*C. stock | −80*C. stock | −80*C. stock |
| Inoculation Prep | plate | plate | plate | plate | plate | seed flask | seed flask |
| Time (hours) | 96 | 96 | 72 | 72-96 | 72 | 48+ | 48+ |
| Temperature | 30*C. | 30*C. | 30*C. | 30*C. | 30*C. | 30*C. | 30*C. |
| Culture Volume (mL) | 50 | 100 | 500 | 500 | 50 | 3100 | 3100 |
| Container Volume (mL) | 250 | 250 | 2000 | 2500 | 250 | 5000 | 5000 |
| Container Type | shake flask | shake flask | shake flask | shake flask | shake flask | bioreactor | bioreactor |
| Container Material | glass | glass | glass | polypropylene | glass | glass | glass |
| Container Shape | | | skinny mouth, | large mouth, | | | |
| | | | large baffles | large baffles | | | |
| Agitation Type | shake | shake | shake | shake | shake | impeller | impeller |
| Feeding Type | batch | batch | batch | batch | fed-batch | batch | fed-batch |
| Feeding Protocol | initial medium | initial medium | initial medium | initial medium | +sucrose @ | initial | +sucrose @ 16 |
| | only | only | only | only | 16 hours | medium only | hours, then |
| | | | | | | | hourly for 8 |
| | | | | | | | hours |
| # Culture | 2 | 1 | 2 | 1 | 1 | 2 | 1 |
| Morphologies | | | | | | | |
| Primary Culture | smooth & | smooth | smooth & | smooth | smooth | smooth & | smooth |
| Phenotype(s) | rough | | rough | | | rough | |

SF = Shake Flask;
BIO = Bioreactor;
B = Batch feeding;
FB = Fed-Batch feeding

### Biophysical Characterization of Culture and Fermentation

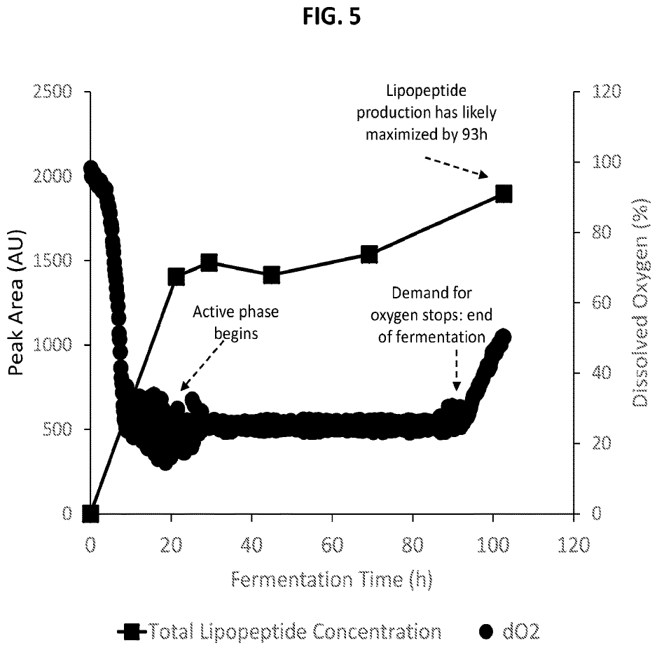

Measurements of dissolved oxygen, lipopeptide production, and pH were obtained from bioreactor cultures over time. As shown in FIG. 5, Dissolved Oxygen (DO₂) levels decrease as a culture begins active growth phase, which correlates to increasing production of lipopeptides. As the culture slows fermentation rate, demand for oxygen stops. Maximum rates of colony growth are typically seen during high oxygen availability; thus, decreasing available DO₂, or increasing oxygen stress, would be one way of slowing down or prolonging the growth phase of a culture.

Table 3 shows TVCs (Total Viable Cell counts) spore counts per Strain 20 variant culture. For all Smooth started cultures, the TVCs were a mixture of R+S, but spores were all Smooth. For all Rough started cultures, the TVCs and spores were Rough.

TABLE 3
Spore counts and Total Viable Cells (TVC)
per culture for Strain 20 variants

| Colony | pH | TVC | Spore |
|---|---|---|---|
| Smooth 1 | 7.34 | 3.60E+09 | 6.25E+09 |
| Smooth 2 | 7.43 | 2.60E+09 | 7.20E+09 |
| Smooth 3 | 7.26 | 5.50E+09 | 6.35E+09 |
| Smooth 4 | 7.19 | 2.55E+09 | 4.80E+09 |
| Smooth 5 | 7.2 | 3.71E+09 | 4.85E+09 |
| Smooth 6 | 7.4 | 2.70E+09 | 6.05E+09 |
| Smooth 7 | 7.35 | 3.50E+09 | 5.25E+09 |
| Smooth 8 | 7.12 | 3.30E+09 | 6.00E+09 |
| Smooth 9 | 7.27 | 3.20E+09 | 5.10E+09 |
| Smooth 10 | 7.36 | 3.30E+09 | 5.40E+09 |
| Smooth 11 | 7.18 | 3.20E+09 | 4.05E+09 |
| Smooth 12 | 6.88 | 4.25E+09 | 5.10E+09 |
| Smooth 13 | 7.26 | 3.55E+09 | 5.90E+09 |
| Smooth 14 | 7.36 | 4.55E+09 | 5.65E+09 |
| Smooth 15 | 7.29 | 5.75E+09 | 6.20E+09 |
| Rough 1 | 6.64 | 3.00E+09 | 5.95E+09 |
| Rough 2 | 6.8 | 2.35E+09 | 6.55E+09 |
| Rough 3 | 7.05 | 3.40E+09 | 3.77E+09 |
| Rough 4 | 8.58 | 2.90E+09 | 1.66E+09 |
| Rough 5 | 6.56 | 5.20E+09 | 6.30E+09 |
| Rough 6 | 8.48 | 3.35E+09 | 1.74E+09 |
| Rough 7 | 6.95 | 4.25E+09 | 3.95E+09 |
| Rough 8 | 7.72 | 1.11E+09 | 2.41E+09 |
| Rough 9 | 8.32 | 4.50E+09 | 2.66E+09 |
| Rough 10 | 6.7 | 4.45E+09 | 7.35E+09 |
| Rough 11 | 7.78 | 1.96E+09 | 3.75E+09 |
| Rough 12 | 8.26 | 2.50E+09 | 2.39E+09 |
| Rough 13 | 7.9 | 2.64E+09 | 5.15E+09 |
| Rough 14 | 6.81 | 6.05E+09 | 8.25E+09 |
| Rough 15 | 6.48 | 1.11E+09 | 6.70E+09 |

R = Rough morphology colony; S = Smooth morphology colony; number after the R or S indicates the variant colony number.

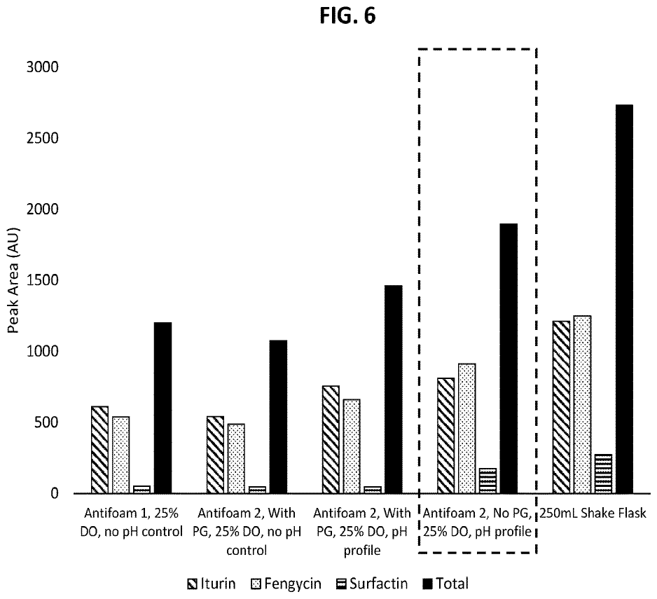

Methods of controlling pH were evaluated to improve the production of lipopeptides during fermentation (correlated to the growth phase of the culture). FIG. 6 depicts differences in total lipopeptide production (in addition to individual levels of Iturin, Fengycin, and Surfactin), under different culture conditions. By using pH as a control strategy, propylene glycol was not required to maintain Oxygen levels, as seen in the dataset surrounded by the dashed box. As seen in Table 4A, controlling pH was the key to obtaining higher activity against several disease targets, due to the improved production of lipopeptide active ingredients. Tables 4B and 4C show that by adjusting oxygen levels via pH control, antifungal activity from scaled-up bioreactor fermentation was better than a commercially-available product, and similar to small-scale shake flask fermentation products.

TABLE 4A
Fusarium assay scores (0-5) for microbes produced
using different bioreactor fermentation conditions

| | | | Fusarium assay scores | | | | |
|---|---|---|---|---|---|---|---|
| BR Run | Conditions | Sample Time (h) | 1X | 5X | 10X | 50X | 100X |
| 1 | Antifoam 2 | 0.20 | 0 | 0 | 0 | 0 | 0 |
| | No pH control | 20.30 | 0 | 0 | 0 | 0 | 0 |
| | 40% DO | 25.10 | 3 | 0 | 0 | 0 | 0 |
| | | 28.50 | 3 | 1 | 0 | 0 | 0 |
| | | 44.30 | 5 | 3 | 1 | 0 | 0 |
| | | 48.10 | 4 | 4 | 3 | 0 | 0 |
| | | 51.00 | 4 | 4 | 4 | 0 | 0 |
| 2 | Antifoam 2 | 0.25 | 0 | 0 | 0 | 0 | 0 |
| | Shake flask pH profile | 20.50 | 3 | 2 | 0 | 0 | 0 |
| | 25% DO | 26.00 | 4 | 3 | 3 | 0 | 0 |
| | | 28.75 | 4 | 4 | 4 | 1 | 0 |
| | | 44.50 | 4 | 4 | 4 | 3 | 3 |
| | | 49.50 | 4 | 4 | 4 | 3 | 3 |
| | | 52.75 | 4 | 4 | 4 | 3 | 3 |
| | | 68.75 | 4 | 4 | 3 | 3 | 3 |
| | | 71.75 | 4 | 3 | 3 | 3 | 3 |
| | | 76.25 | 4 | 3 | 3 | 3 | 3 |
| | | 93.00 | 4 | 3 | 3 | 3 | 3 |
| 3 | Antifoam 2 | 0.17 | 0 | 0 | 0 | 0 | 0 |
| (Repeat of | Shake flask pH profile | 18.17 | 3 | 0 | 0 | 0 | 0 |
| BR 2) | 25% DO | 27.50 | 4 | 4 | 4 | 2 | 0 |
| | | 42.25 | 4 | 4 | 4 | 4 | 3 |
| | | 47.83 | 4 | 4 | 4 | 4 | 3 |
| 4 | Antifoam 2 | 0.17 | 0 | 0 | 0 | 0 | 0 |
| | Shake flask pH profile | 18.17 | 3 | 0 | 0 | 0 | 0 |
| | 40% DO | 27.50 | 4 | 4 | 4 | 2 | 1 |
| | | 42.25 | 4 | 4 | 4 | 4 | 3 |
| | | 47.83 | 4 | 4 | 4 | 4 | 3 |

DO = Dissolved Oxygen; 0 = least control, 5 = most control.

TABLE 4B
Fusarium oxysporum assay scores (0-5) for microbes
produced using different fermentation conditions

| | Fusarium oxysporum score (0-5) at different dilutions | | | | | | | |
|---|---|---|---|---|---|---|---|---|
| Sample | 1X | 10X | 50X | 100X | 200X | 300X | 400X | 500X |
| Shake flask, no antifoam | 4 | 4 | 3 | 4 | 4 | 4 | 3 | 3 |
| Antifoam 2, pH profile 1, | 4 | 4 | 3 | 3 | 3 | 3 | 2 | 1 |
| 25% DO | | | | | | | | |
| Commercial microbe | 5 | 4 | 3 | 2 | 1 | 1 | 0 | 0 |
| supernatant | | | | | | | | |

TABLE 4C
Botrytis cinera assay scores (0-5) for microbes
produced using different fermentation conditions

| | Botrytis cinera score (0-5) at different dilutions | | | | | | | |
|---|---|---|---|---|---|---|---|---|
| Sample | 1X | 10X | 50X | 100X | 200X | 300X | 400X | 500X |
| Shake flask, no antifoam | 5 | 5 | 5 | 5 | 4 | 3 | 3 | 2 |
| Antifoam 2, pH profile 1, | 5 | 5 | 5 | 5 | 4 | 3 | 2 | 2 |
| 25% DO | | | | | | | | |
| Commercial microbe | 5 | 5 | 3 | 2 | 1 | 1 | 0 | 0 |
| supernatant | | | | | | | | |

Based on the evidence, conditions that reduce nutrient stress and allowing cultures to have a longer growth phase for lipopeptide production, for example by promoting greater nutrient availability, decreasing cellular crowding, and/or longer and/or slower growth phases, for example by controlling the oxygen availability, pH, and/or other conditions, encourage the production of lipopeptides that are capable of conferring positive benefits for plant health.

### Example 3: Identification and Confirmation of DNA Changes in Morphological Variants

Using DNA isolation, next-gen library assembly and sequencing, and bioinformatic analysis, the exact nucleic acid differences between two morphological variants of the bacterial Strain 20 were determined, and verified that contamination during fermentation was not the source of these variants.

### DNA Isolation

A “Smooth” Strain 20 plate and a “Rough” Strain 20 plate were obtained. From each plate, 10 colonies were selected (1S-10S and 1R-10R; S=smooth morphology, R=rough morphology). The colonies were grown in a 96-well block with 1 ml of TSB for 48 hours at 30 C. DNA was extracted using the Qiagen DNeasy Powersoil HTP 96 Kit, centrifuge Protocol (DNeasy PowerSoil HTP 96 Kit Handbook, revision August 2019, pages 8-10):
1. Remove Square Well Mat from a PowerBead Plate. Add up to 0.25 g of soil sample. Note: Avoid cross contamination between sample wells. This is an appropriate stopping point. You can store the PowerBead Plate at 2-8° C. covered with the Square Well Mat.
2. Add 750 μl of PowerBead Solution to the wells of the PowerBead Plate.
3. Add 60 μl of Solution C1. Secure the Square Well Mat tightly to the plate.
4. Place PowerBead Plate with mat securely fastened between 2 Adapter Plates (cat. no. 11990) on a 96 well plate shaker or a TissueLyser II (cat. no. 85300).
5. Shake at speed 20 Hz for 10 min. Re-orient plates so that the side that was closest to the machine body is now furthest from it and shake again at speed 20 Hz for 10 min.
6. Centrifuge at room temperature for 6 min at 4500×g.
7. Discard the Square Well Mat. Transfer the supernatant to a clean 1 ml Collection Plate. Note: The supernatant may still contain some soil particles.
8. Add 250 μl of Solution C2.
9. Apply Sealing Tape to plate. Vortex for 5 s. Incubate at 2-8° C. for 10 min.
10. Centrifuge the plate at room temperature for 6 min at 4500×g. Discard Sealing Tape.
11. Avoiding the pellet, transfer entire volume of supernatant to a new 1 ml Collection Plate.
12. Apply Sealing Tape to plate and repeat steps 10-11 once. Then move on to step 13.
13. Add 200 μl of Solution C3 and repeat steps 9-11 once. Then apply Sealing Tape to the plate and centrifuge again at room temperature for 6 min at 4500×g.
14. Transfer no more than 650 μl of supernatant to a 2 ml Collection Plate.
15. Add 650 μl of Solution C4 to each well of the plate. Repeat (to add 1300 μl total). Note: You can pause here and store the samples covered with Sealing Tape at 2-8° C.
16. Pipet samples up and down to mix. Place a spin plate onto an S-Block.
17. Load approximately 650 μl into each well of the spin plate and seal the plate with an AirPore Tape Sheet.
18. Centrifuge at room temperature for 3 min at 4500×g. Discard the flow-through and place the spin plate back on the same S-Block. Discard the AirPore Tape Sheet.
19. Repeat steps 17 and 18 until all the supernatant has been processed. Discard the final flow-through.
20. Place the spin plate back on the same S-Block.
21. Add 500 μl of Solution C5-D to each well of the spin plate and seal the plate with an AirPore Tape Sheet.
22. Centrifuge at room temperature for 3 min at 4500×g. Discard the flow-through and place the spin plate back on the same S-Block. Seal with an AirPore Tape Sheet.
23. Centrifuge again at room temperature for 5 min at 4500×g. Discard flow-through.
24. Carefully place the spin plate onto Racked Elution Microtubes. Discard the AirPore Tape Sheet.
25. Allow to air dry for 10 min at room temperature.
26. Add 100 μl of Solution C6 to the center of each well. Seal plate with an AirPore Tape Sheet.
27. Centrifuge at 4500×g for 3 min at room temperature. Discard the AirPore Tape Sheet.
28. Seal Elution Microtubes with the Caps provided. The DNA is now ready for downstream applications.

The protocol was followed with slight changes to step 5. Step 5 was performed for 7 minutes instead of 10 minutes. Once the DNA isolation was completed, DNA quantification was done on the colonies using the PicoGreen dsDNA reagent kit. The DNA obtained from the 20 colonies averaged around 10 ng/ul, with the exception of R5 which did not yield any DNA due to poor elution. Despite the poor elution of R5, all 20 colonies were included in the library preparation.

### Library Construction and Whole Genome Sequencing

Libraries were constructed using the iGenomix Riptide High Throughput Rapid DNA Library Prep protocol. The protocol was completed with an alteration to the PCR program in step 1M. The program includes several rounds of amplification to compensate for low DNA yields from the DNA isolation step. Once completed, the genomic sequencing libraries are sent to Admera Health for next generation sequencing. Sequencing was performed on an Illumina HiSeq with PE150 with 10×PhiX.

### Variant Calling and Confirmation

Prior to data delivery, reads associated with PhiX were removed from the dataset. Samples with at least 50× genome coverage moved forward to the variant calling pipeline. Raw Illumina reads were demultiplexed with fgbio 0.6.1 and trimmed to a Q score of 25 with Trimmomatic v38. Cleaned reads were mapped to the pacbio-illumina hybrid genome assembly using minimap2 2.17. Sam files were converted to Bam files and sorted with Samtools 1.9. Consensus sequences were identified, and variants called with bcftools 1.9. Due to poor genome coverage, R5 was removed from the analysis. VCF files were mapped to the strain hybrid genome assembly and viewed in Geneious Prime build 2021 Mar. 12.

To confirm results of the Rough vs. Smooth variant analysis, Sanger sequencing was performed in the region of interest. Primers were designed to amplify the region of interest in the DNA from 19 of the 20 colonies, R5 was dropped due to low DNA yield and poor Illumina results. PCR was run on these samples. Once completed the PCR products were run on an agarose gel to confirm that the expected product was correctly amplified. After gel confirmation, the PCR products were sequenced. Variants were confirmed with the alignment of Sanger sequence reads to the Strain 20 reference genome/vcf file assembly in Geneious.

### Results

Genomic sequences of: Strain 20 hybrid assembly rghR gene is given as SEQID NO: 3; Strain 20 hybrid assembly region of interest (variation region) is given as SEQID NO: 4; regions of interest (variation region) for Strain 20 Smooth Variants 1-10 are given as SEQID NOs: 5-14, respectively; and regions of interest (variation region) for Strain 20 Rough Variants 1-5 and 6-10 are given as SEQID NOs: 15-23, respectively.

Sample Strain20_R5 was removed from the sample set due to 0.5× total genomic coverage prior to trimming which is significantly below quality thresholds. Remaining samples were analyzed with the variant calling pipeline with an average of 8 nucleotides differing between the reference sequence and the experimental samples with no significant difference between Rough and Smooth colony morphologies, 5±4 and 9±6, respectively, with Strain20_S1 containing 48 total SNPs.

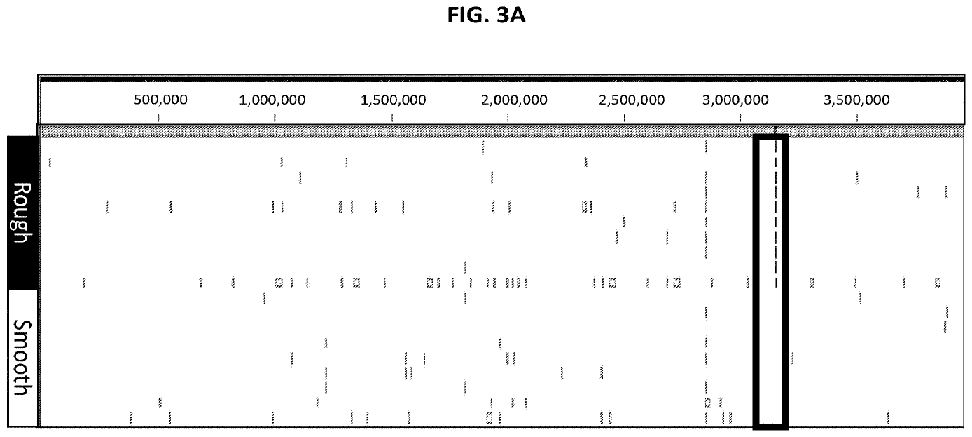

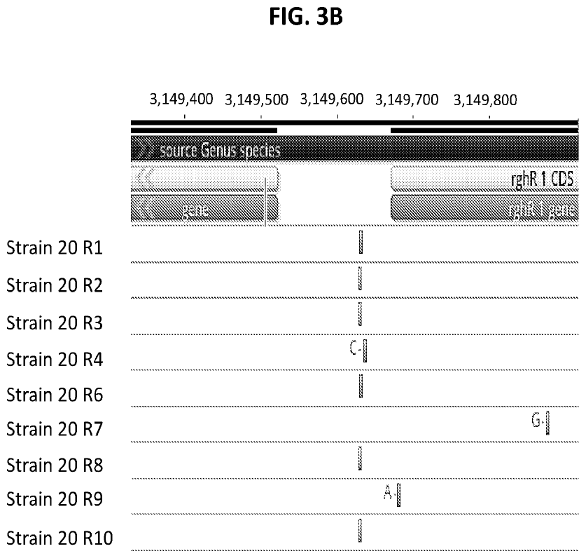

Closer examination of the Strain 20 Rough vs. Smooth variants revealed a concentration of SNPs in the promotor or coding region of the rghR gene which was absent from the samples exhibiting the smooth colony morphology phenotype (FIGS. 3A and 3B, Table 5).

Sanger confirmation sequences covered bases 3,149,612 through 3,150,100 for all rough and smooth samples with the exception of Strain20_S10, which did not align to the reference due to poor sequence quality. Each rough colony morphology rghR associated SNP identified in the variant calling analysis was confirmed and all samples originating from smooth colony morphology samples were confirmed to have sequences which match the reference (wildtype).

Conserved SNPs were in the rghR promoter/gene of all of the Rough isolates, and none of the Smooth isolates. For one of the coding region mutations, TTC→TA changes were neutral, non-polar amino acid (phenylalanine) to a charged, polar amino acid (lysine). For another coding region mutation, TAC→TGC shifted a tyrosine residue to a cysteine residue. In both cases, a hydrophobic aromatic amino acid was changed to a non-aromatic hydrophilic amino acid.

The rghR promoter appears to be a bidirectional promoter, and further analysis suggests that the rghR gene appears to be a helix-turn-helix regulator.

Through DNA isolation, whole genome sequencing, bioinformatics, and Sanger sequencing we have definitively shown that a Bacillus created 2 different morphologies (Smooth and Rough) due DNA changes in the locus rghR (coding and regulatory regions). There were no other significant DNA changes between these two morphological variants, and there was no contamination detected during fermentation.

TABLE 5A
Genomic sequence identification of SNPs correlated
to “Rough” bacterial phenotypes

| | | | Genomic | | |
|---|---|---|---|---|---|
| Strain 20 | | Total | Location | | |
| Sample | Phenotype | SNPs | (base) | Type | Locus/Gene |
| R1 | Rough | 3 | 3149632 | Deletion | rghR promoter |
| R2 | Rough | 5 | 3149631 | Deletion | rghR promoter |
| R3 | Rough | 5 | 3149631 | Deletion | rghR promoter |
| R4 | Rough | 5 | 3149637 | T → C | rghR promoter |
| R6 | Rough | 16 | 3149632 | Deletion | rghR promoter |
| R7 | Rough | 3 | 3129877 | A → G | rghR gene |
| R8 | Rough | 4 | 3149631 | Deletion | rghR promoter |
| R9 | Rough | 2 | 3149683 | C → A | rghR gene |
| R10 | Rough | 2 | 3149631 | Deletion | rghR promoter |

### Example 4: Lipopeptide Profile Analysis

Lipopeptide profiles were obtained for the Whole Cell Broth (WCB) and Supernatant for a Bacillus strain, Strain 20. 15 Rough and 15 Smooth colonies were isolated after fermentation, and grown separately in optimized media in shake flasks.

Supernatant material and WCB material were analyzed according to the following HPLC protocol: C18 stationary phase with a particle size of 5 micrometers and pore size 120A was prepared in a column of 4.6 mm diameter and 150 mm length, at a temperature of 25 degrees Celsius. A gradient runtime of 30 minutes at a flow rate of 0.8 mL/min was conducted, with a mobile phase solvent A (water with 0.01% trifluoroacetic acid) and mobile phase solvent B (acetonitrile with 0.01% trifluoroacetic acid), using a gradient of: 0-3 min: 40% solvent B; 3-8 min: 40-50% solvent B linear gradient; 8-13 min: 50-80% solvent B linear gradient; 13-30 min: 80-100% solvent B linear gradient. Eluate was analyzed at 205 nm.

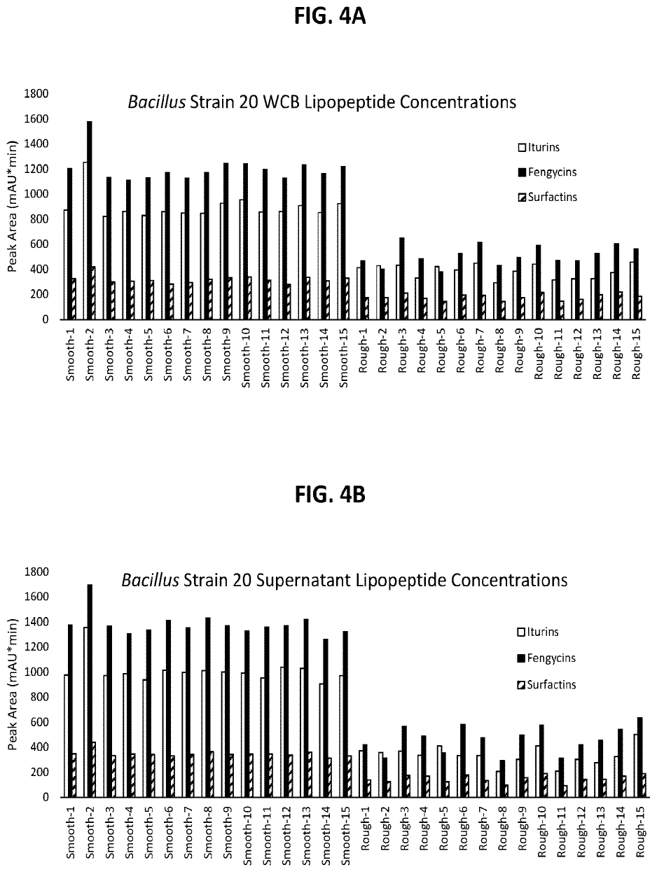

As shown in FIGS. 4A and 4B, both the WCB and Supernatant showed different amounts of each of Iturins Fengycins, and Surfactins, as well as different ratios of each.

Using the following calculations, the approximate concentrations of each lipopeptide can be estimated:

$$Iturins Concentration ( mg / L ) = 1.1925 * ( Peak Area ) - 36.778$$

$$Surfactins Concentration ( mg / L ) = 0.6432 * ( Peak Area ) - 16.798$$

Smooth colonies produced over 520 mg/L Iturins and over 150 mg/L of Surfactins. Smooth colonies had a peak ratio of Fengycins:Surfactins of at least 6.1, and a peak ratio of Iturin:Fengycin:Surfactin of less than 0.0028.

TABLE 6
Lipopeptide profiles of different Strain 20 variant cultures

| | Iturins | | Fengycins | Surfactins | | Peak | Peak | Peak | Peak |
|---|---|---|---|---|---|---|---|---|---|
| | Peak | [Iturin] | Peak | Peak | [Surfactin] | Ratios | Ratios | Ratios | Ratios |
| Type | Area | mg/L | Area | Area | mg/L | I:F | I:S | F:S | I:F:S |
| Rough-13 | 325.27 | 351.10 | 526.99 | 197.40 | 110.17 | 0.61722 | 1.64777 | 4.78350 | 0.00313 |
| Rough-14 | 373.16 | 408.22 | 602.24 | 220.27 | 124.88 | 0.61963 | 1.69415 | 4.82262 | 0.00281 |
| Rough-3 | 432.38 | 478.84 | 646.80 | 209.93 | 118.23 | 0.66849 | 2.05968 | 5.47087 | 0.00318 |
| Rough-11 | 316.12 | 340.20 | 469.83 | 146.57 | 77.47 | 0.67284 | 2.15687 | 6.06447 | 0.00459 |
| Rough-8 | 291.36 | 310.67 | 429.33 | 144.18 | 75.94 | 0.67864 | 2.02086 | 5.65383 | 0.00471 |
| Rough-4 | 331.39 | 358.41 | 482.49 | 168.97 | 91.88 | 0.68684 | 1.96123 | 5.25099 | 0.00406 |
| Rough-12 | 324.69 | 350.42 | 466.43 | 160.89 | 86.68 | 0.69612 | 2.01816 | 5.38090 | 0.00433 |
| Smooth-11 | 856.42 | 984.50 | 1194.55 | 312.02 | 183.90 | 0.71694 | 2.74472 | 6.49583 | 0.00230 |
| Smooth-8 | 845.67 | 971.68 | 1171.69 | 322.03 | 190.33 | 0.72175 | 2.62607 | 6.15606 | 0.00224 |
| Smooth-1 | 872.36 | 1003.51 | 1203.96 | 324.39 | 191.85 | 0.72458 | 2.68924 | 6.27551 | 0.00223 |
| Smooth-3 | 821.69 | 943.09 | 1133.35 | 297.91 | 174.82 | 0.72501 | 2.75819 | 6.48305 | 0.00243 |
| Rough-7 | 449.91 | 499.74 | 615.21 | 190.07 | 105.46 | 0.73132 | 2.36707 | 5.83381 | 0.00385 |
| Smooth-14 | 852.23 | 979.50 | 1163.45 | 306.35 | 180.25 | 0.73250 | 2.78187 | 6.45476 | 0.00239 |
| Smooth-5 | 828.56 | 951.28 | 1129.41 | 310.61 | 182.99 | 0.73362 | 2.66753 | 6.17212 | 0.00236 |
| Smooth-6 | 859.88 | 988.63 | 1171.43 | 282.70 | 165.03 | 0.73404 | 3.04169 | 7.09819 | 0.00260 |
| Smooth-13 | 910.01 | 1048.41 | 1231.06 | 335.06 | 198.72 | 0.73921 | 2.71593 | 6.19511 | 0.00221 |
| Smooth-9 | 927.16 | 1068.86 | 1242.83 | 333.53 | 197.73 | 0.74601 | 2.77982 | 6.28548 | 0.00224 |
| Rough-10 | 441.02 | 489.14 | 590.81 | 215.87 | 122.05 | 0.74647 | 2.04304 | 4.84082 | 0.00346 |
| Rough-6 | 395.15 | 434.44 | 526.19 | 194.33 | 108.19 | 0.75096 | 2.03340 | 4.86337 | 0.00386 |
| Smooth-7 | 849.19 | 975.88 | 1127.05 | 293.19 | 171.78 | 0.75346 | 2.89640 | 6.56103 | 0.00257 |
| Smooth-15 | 923.26 | 1064.21 | 1221.07 | 331.14 | 196.19 | 0.75610 | 2.78811 | 6.22388 | 0.00228 |
| Smooth-12 | 862.98 | 992.33 | 1128.40 | 279.97 | 163.28 | 0.76479 | 3.08245 | 6.91096 | 0.00273 |
| Smooth-10 | 954.37 | 1101.30 | 1239.90 | 339.71 | 201.70 | 0.76971 | 2.80940 | 6.14722 | 0.00227 |
| Rough-9 | 384.19 | 421.37 | 494.84 | 173.27 | 94.65 | 0.77640 | 2.21731 | 5.22813 | 0.00448 |
| Smooth-4 | 863.39 | 992.82 | 1109.07 | 306.13 | 180.10 | 0.77848 | 2.82040 | 6.15805 | 0.00254 |
| Smooth-2 | 1252.49 | 1456.82 | 1578.63 | 417.27 | 251.59 | 0.79340 | 3.00162 | 6.27460 | 0.00190 |
| Rough-15 | 459.10 | 510.70 | 564.19 | 184.61 | 101.95 | 0.81372 | 2.48679 | 5.53425 | 0.00441 |
| Rough-1 | 413.48 | 456.29 | 466.67 | 173.33 | 94.69 | 0.88602 | 2.38553 | 4.92861 | 0.00511 |
| Rough-2 | 427.80 | 473.37 | 401.36 | 175.86 | 96.31 | 1.06587 | 2.43260 | 4.16717 | 0.00606 |
| Rough-5 | 419.84 | 463.88 | 379.18 | 144.50 | 76.15 | 1.10723 | 2.90540 | 4.97961 | 0.00766 |

### Example 5: In Planta Nematode Bioassay Activity

Nematocidal activity of Rough and Smooth colonies of Bacillus Strain 20 was evaluated in tomato plants. 15 Rough and 15 Smooth colonies were harvested after fermentation, and grown separately in optimized media. Whole cell broths were applied to tomato plants, which were inoculated with nematodes. Results are shown in Tables 6-8. Overall, Smooth colonies provided greater nematode control than Rough colonies (UTC=UnTreated Control/not infected and not inoculated; IC=Infected Control/infected with nematodes but not inoculated with a Bacillus strain).

TABLE 6
Reduction in average egg masses per gram of dry root (lower number =
greater reduction, higher number = less reduction)

| Bacillus Strain Variant #/ | Average egg masses/g dry root compared |
|---|---|
| Morphology (S or R) | to the UnTreated Control (UTC) |
| 7 S pH 7.34 | 1.46% |
| 9 S pH 7.26 | 16.43% |
| 10 S pH 7.19 | 3.28% |
| 11 S pH 7.2 | −14.24% |
| 12 S pH 7.4 | −33.57% |
| 13 S pH 7.35 | −0.66% |
| 15 S pH 7.27 | 3.34% |
| 16 S pH 7.36 | −5.86% |
| 17 S pH 7.18 | −39.17% |
| 19 S pH 7.26 | −21.55% |
| 20 S pH 7.36 | −16.80% |
| 21 S pH 7.29 | −1.32% |
| 7 S heat treated | 2.46% |
| 22 R pH 6.64 | −2.47% |
| 23 R pH 6.8 | −6.23% |
| 25 R pH 8.58 | −3.36% |
| 26 R pH 6.56 | −28.74% |
| 27 R pH 8.48 | 30.48% |
| 29 R pH 7.72 | 16.78% |
| 30 R pH 8.32 | −0.88% |
| 31 R pH 6.7 | −7.48% |
| 32 R pH 7.78 | 44.04% |
| 33 R pH 8.26 | 24.91% |
| 35 R pH 6.81 | −5.01% |
| 36 R pH 6.48 | 21.96% |
| 35 R low heat treated | 29.05% |
| 24 R mid | 12.93% |
| 25 R high heat treated | −2.67% |
| 1147 plate | 11.41% |
| Untreated Control | −99.74% |

TABLE 7
Average shoot dry weight

| | Average of shoot dry |
|---|---|
| Bacillus Strain Variant #/ | mass as compared to |
| Morphology (S or R) | the UTC |
| 7 S pH 7.34 | 12.49% |
| 9 S pH 7.26 | 11.64% |
| 10 S pH 7.19 | 23.63% |
| 11 S pH 7.2 | 12.03% |
| 12 S pH 7.4 | 32.37% |
| 13 S pH 7.35 | −1.94% |
| 15 S pH 7.27 | 16.65% |
| 16 S pH 7.36 | 3.71% |
| 17 S pH 7.18 | 14.55% |
| 19 S pH 7.26 | 9.29% |
| 20 S pH 7.36 | 7.71% |
| 21 S pH 7.29 | −2.08% |
| 7 S heat treated | 0.33% |
| 22 R pH 6.64 | −2.43% |
| 23 R pH 6.8 | 2.99% |
| 25 R pH 8.58 | −9.09% |
| 26 R pH 6.56 | 41.03% |
| 27 R pH 8.48 | −21.39% |
| 29 R pH 7.72 | −17.43% |
| 30 R pH 8.32 | 11.68% |
| 31 R pH 6.7 | 21.20% |
| 32 R pH 7.78 | −11.66% |
| 33 R pH 8.26 | −2.49% |
| 35 R pH 6.81 | −1.36% |
| 36 R pH 6.48 | −19.20% |
| 35 R low heat treated | −3.79% |
| 24 R mid | 9.97% |
| 25 R high heat treated | −3.13% |
| 1147 plate | 15.21% |
| IC | 10.04% |
| UTC | |

TABLE 8
Average root dry mass (grams)

| Bacillus Strain Variant #/ | Average of root dry |
|---|---|
| Morphology (S or R) | mass (grams) |
| 7 S pH 7.34 | 0.3270 |
| 9 S pH 7.26 | 0.2975 |
| 10 S pH 7.19 | 0.3037 |
| 11 S pH 7.2 | 0.3583 |
| 12 S pH 7.4 | 0.4123 |
| 13 S pH 7.35 | 0.3013 |
| 15 S pH 7.27 | 0.3363 |
| 16 S pH 7.36 | 0.3260 |
| 17 S pH 7.18 | 0.4112 |
| 19 S pH 7.26 | 0.3437 |
| 20 S pH 7.36 | 0.3307 |
| 21 S pH 7.29 | 0.2707 |
| 7 S heat treated | 0.2797 |
| 22 R pH 6.64 | 0.3197 |
| 23 R pH 6.8 | 0.3015 |
| 25 R pH 8.58 | 0.3095 |
| 26 R pH 6.56 | 0.4702 |
| 27 R pH 8.48 | 0.2662 |
| 29 R pH 7.72 | 0.3005 |
| 30 R pH 8.32 | 0.3103 |
| 31 R pH 6.7 | 0.3648 |
| 32 R pH 7.78 | 0.2708 |
| 33 R pH 8.26 | 0.3427 |
| 35 R pH 6.81 | 0.3212 |
| 36 R pH 6.48 | 0.2433 |
| 35 R low heat treated | 0.3360 |
| 24 R mid | 0.3348 |
| 25 R high heat treated | 0.2782 |
| 1147 plate | 0.3432 |
| IC | 0.2977 |
| UTC | 0.3100 |

In summary, Bacillus Smooth morphology colonies demonstrated better egg mass reduction compared to Rough, and better shoot dry weight compared to Rough. Rough morphology colonies demonstrated less egg mass reduction compared to Smooth, and seemed to negatively affect shoot dry weight.

### Example 6: In Planta Fungus Bioassay Activity

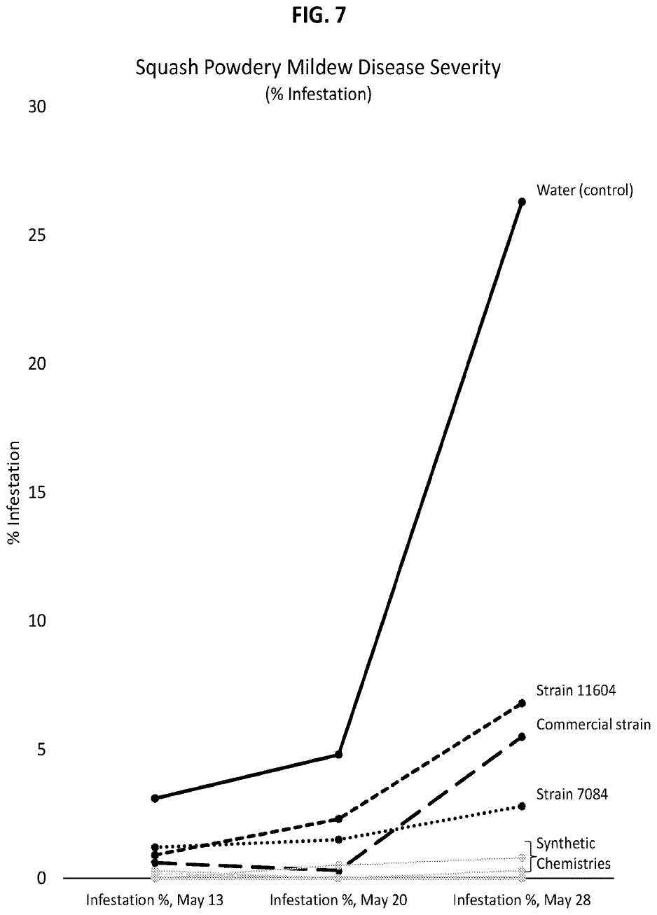

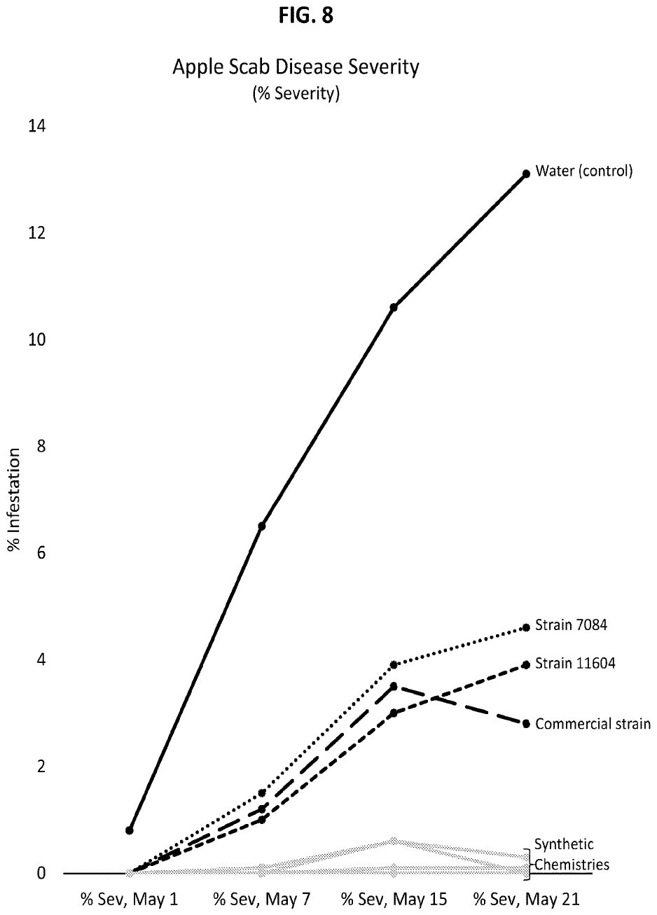

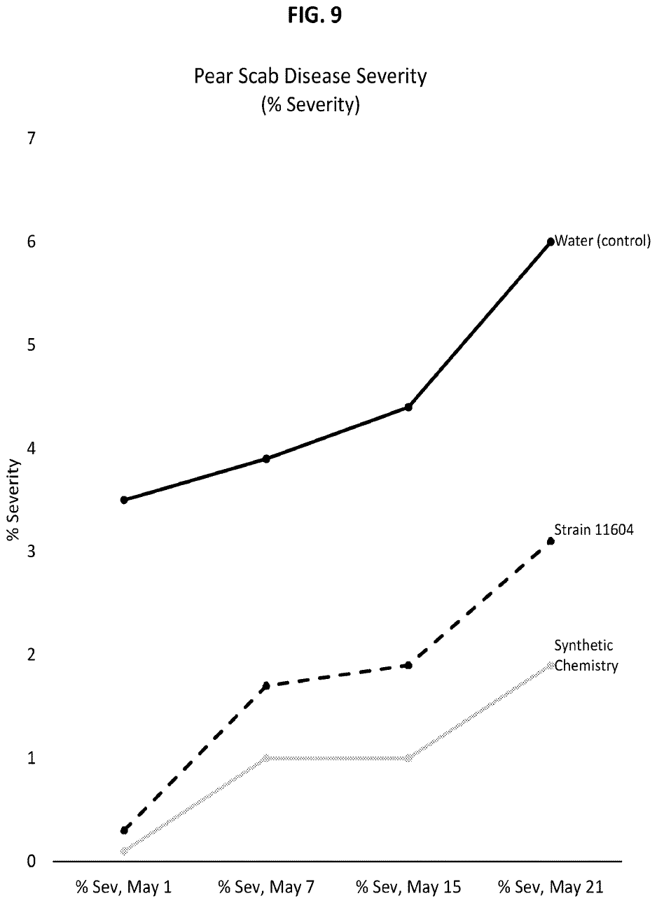

Strains 7084 and 11604 were produced in a bioreactor using batch feeding protocols, resulting in Smooth colonies. Activity was evaluated against three different fungal disease targets: squash powdery mildew, apple scab, and pear scab. Data are shown in FIGS. 7-9.

Squash Powdery Mildew was better controlled with Strains 7084 and 11604 for the first several weeks than with a commercially-available microbial product. Apple Scab Disease was controlled better than with a commercially-available microbial product for the first several weeks. Pear Scab Disease was controlled across several weeks as compared to a water-control product.

1. A method of producing a bacterial composition capable of conferring benefit to a target, the method comprising:
(a) obtaining a source bacterium of the taxonomic family Bacillaceae;
(b) placing the source bacterium in a fermentation medium;
(c) incubating the source bacterium under one or more condition(s) that promote an elongation of the logarithmic or exponential growth phase of the bacterium, to produce an incubated material;
wherein the incubated material comprises a population of bacteria that comprises a Smooth morphology phenotype at a level of at least 10 times greater than the level of any other morphology phenotype; and
(d) removing a product from the incubated material of (c);
wherein the product of (d) comprises a composition of benefit to a target.

2. The method of claim 1, wherein the one or more condition(s) of (c) is (are) selected from the group consisting of: increased oxygen stress, decreased nutrient stress, decreased agitation, decreased CFUs per unit area, increased CFUs per unit area, altered temperature, altered pressure, modified pH, reduced water availability, radiation, sonication, introduction of a chemical agent, presence or absence of a drying agent, solid fermentation media, liquid fermentation media, and introduction of a biological agent.

3. The method of claim 1, wherein the source bacterium comprises an inducible promoter, wherein the inducible promoter is heterologous to the source bacterium.

4. (canceled)

5. The method of claim 1, wherein the product of (d) comprises bacterial cells.

6. The method of claim 1, wherein the product of (d) comprises bacterial cell broth and/or a substantially cell-free supernatant of the incubated material of (c).

7. (canceled)

8. (canceled)

9. The method of claim 1, wherein the source bacterium produces a plurality of different lipopeptides selected from the group consisting of: a Fengycin, an Iturin at a concentration of at least 520 mg/L, a Surfactin at a concentration of at least 150 mg/L, and any plurality and/or combination of the preceding.

10. The method of claim 1, wherein the source bacterium produces a composition that comprises a Fengycin (F), an Iturin (I), and a Surfactin(S), wherein the ratio of I:F:S is less than 0.00280.

11. The method of claim 1, wherein the source bacterium produces a composition that comprises a Fengycin (F), an Iturin (I), and a Surfactin(S), wherein the ratio of F:S is greater than 6.10.

12. The method of claim 1, wherein the product of (d) comprises a substantially purified bacterial-produced composition, wherein the substantially-purified bacterial produced composition comprises a plurality of different lipopeptides including a Fengycin, an Iturin at a concentration of at least 520 mg/L, a Surfactin at a concentration of at least 150 mg/L, and any plurality and/or combination of the preceding.

13. (canceled)

14. (canceled)

15. The method of claim 12, wherein the substantially-purified bacterial produced composition comprises a Fengycin (F), an Iturin (I), and a Surfactin(S), wherein the ratio of I:F:S is less than 0.00280.

16. The method of claim 12, wherein the substantially-purified bacterial produced composition comprises a Fengycin (F), an Iturin (I), and a Surfactin(S), wherein the ratio of F:S is greater than 6.10.

17. The method of claim 1, further comprising placing the incubated material, or composition derived therefrom, in a formulation medium; wherein the formulation medium comprises at least one compound selected from the group consisting of a compatible carrier, a tackifier, a microbial stabilizer, a fungicide, an antibacterial agent, an herbicide, a nematicide, an insecticide, a plant growth regulator, a rodenticide, and a nutrient.

18. (canceled)

19. The method of claim 1, applying the product of (d) to the target or a portion thereof.

20. The method of claim 1, wherein the target is a plant or plant element.

21. The method of claim 20, wherein the plant element comprises seed tissue, leaf tissue, root tissue, or any plurality or combination of the preceding.

22. (canceled)

23. (canceled)

24. The method of claim 1, further comprising incubating the product and/or target under conditions that promote maintenance, growth, and/or reproduction.

25. The method of claim 1, wherein the benefit is bioactivity against a pest or pathogen.

26. The method of claim 25, wherein the pest is a nematode.

27. The method of claim 25, wherein the pathogen is a fungus.

28. The method of claim 20, wherein the benefit to the plant or plant element is an improvement in health, yield, vigor, disease resistance, pest resistance, viability, reproduction, or growth.

29. The method of claim 1, wherein the source bacterium is of the taxonomic genus Bacillus or Lysinibacillus.

30. A composition comprising a microbial population of Bacillaceae microbes and a formulation component, wherein at least one of the microbes of the population comprises a genetic fingerprint comprising the following nucleotide motifs within 1000 nucleotides of an rghR gene, at position numbers relative to an alignment against SEQID NO: 4:
(a) AAAACATACTA at positions 105-113;
(b) GCTTCG at positions 155-160; and
(c) GATACA at positions 350-355;
wherein the eukaryotic cell and the microbial population are heterologously disposed.

31. A composition comprising a substantially pure population of Bacillacea microbes and a formulation component, wherein at least 10% of the substantially pure population is present as a Smooth morphology.

32. The composition of claim 30 or claim 31, wherein the substantially pure population produces at least one composition selected from the group consisting of:
(a) Iturins at a concentration of at least 520 mg/L;
(b) Surfactins at a concentration of a least 150 mg/L;
(c) a ratio of Iturins (I) to Fengycins (F) to Surfactins (S) of less than 0.00280; and
(d) a ratio of Fengycins (F) to Surfactins(S) of greater than 6.10.

33. The composition of claim 30 or claim 31, wherein the microbes are of genus Bacillus.

34. The composition of claim 30 or claim 31, further comprising a eukaryotic cell.

35. The composition of claim 34, wherein the eukaryotic cell is from a plant.

36. The composition of claim 30 or claim 31, further comprising a seed, a leaf, a root, and/or a whole plant.

37. The composition of claim 36, wherein the composition is placed in soil.

38. The composition of claim 37, wherein a plurality of said compositions are placed in a regular pattern with substantially equal spacing between each of the compositions.

39. The composition of claim 36, wherein the seed, leaf, root, and/or whole plant comprises a transgene.

40. The composition of claim 30 or claim 31, further comprising a fungicide, a nematicide, a bactericide, an insecticide, an herbicide, or any combination thereof.

41. The composition of claim 30 or claim 31, wherein the formulation component is selected from the group consisting of: a compound that improves the stability of the microbe, a preservative, a carrier, a surfactant, an anticomplex agent, and any combination thereof.

42. The composition of claim 30 or claim 31, further comprising at least one additional species of microbe.

43. A method of improving the healthy, yield, or vigor of a plant, the method comprising:
(a) placing the plant or plant element thereof in a medium that promotes growth and/or reproduction;
(b) applying to the plant or plant element thereof a heterologous composition, selected from the group consisting of:
i. a bacterium comprising the following motifs within 1000 nucleotides of an rghR gene, at position numbers relative to an alignment against SEQID NO: 4: AAAACATACTA at positions 105-113; GCTTCG at positions 155-160; and GATACA at positions 350-355;
ii. a bacterium producing any one or more of the following: Iturins at a concentration of at least 520 mg/L; Surfactins at a concentration of a least 150 mg/L; a ratio of Iturins (I) to Fengycins (F) to Surfactins(S) of less than 0.00280; and a ratio of Fengycins (F) to Surfactins(S) of greater than 6.10; and
iii. a bacterium exhibiting a Smooth colony morphology at a rate of at least 10 times that of any other colony morphology;
(c) assessing at least one parameter of health, yield, or vigor of the plant, wherein the at least one parameter is improved as compared to an isoline plant not having applied the heterologous composition of (b).

44. The method of claim 43, further comprising a heterologous promoter.

45. The method of claim 44, wherein the heterologous promoter is inducible.
